(19) United States
(12) Patent Application Publication
(10) Pub. No.: US 2025/0355000 A1
(43) Pub. Date: Nov. 20, 2025

(54) URINARY BRANCHED-CHAIN AMINO ACIDS (UBCAAS) AS INSULIN RESISTANCE BIOMARKERS

(71) Applicants: SKILLCELL, JARRY CEDEX (FR); CENTRE NATIONAL DE LA RECHERCHE SCIENTIFIQUE, PARIS (FR); UNIVERSITE DE MONTPELLIER, MONTPELLIER (FR); CENTRE HOSPITALIER UNIVERSITAIRE DE MONTPELLIER, MONTPELLIER (FR)

(72) Inventors: Francisco SANTOS SCHNEIDER, Montpellier (FR); Franck MOLINA, Les Matelles (FR); Eric RENARD, Saint Clement de Rivière (FR)

(73) Assignees: SKILLCELL, JARRY CEDEX (FR); CENTRE NATIONAL DE LA RECHERCHE SCIENTIFIQUE, PARIS (FR); UNIVERSITE DE MONTPELLIER, MONTPELLIER (FR); CENTRE HOSPITALIER UNIVERSITAIRE DE MONTPELLIER, MONTPELLIER (FR)

(21) Appl. No.: 18/871,084

(22) PCT Filed: Jun. 2, 2023
(86) PCT No.: PCT/EP2023/064871

§ 371 Date: Dec. 2, 2024

(30) Foreign Application Priority Data
Jun. 2, 2022 (EP) 22177090.2

(51) Int. Cl. G01N 33/68; C12N 9/02; C12N 9/04; G01N 1/34

(52) U.S. Cl. CPC G01N 33/6893; C12N 9/0006; C12N 9/0063; G01N 1/34; C12Y 101/03004; C12Y 110/03003; G01N 2800/042; G01N 2800/32; G01N 2800/50

(57) ABSTRACT

The present invention is directed to method of determining whether a subject is at risk of developing insulin resistance, particularly for advance alert of T2D and or CVD onset in obese and non-obese subject, by detecting the branched-chain amino acids (BCAAs) present in an urine sample (uBCAAs) of the subjects. The present invention also relates to a method for determining the need of a dietary/nutritional supplement for a subject involving said uBCAAs biomarkers. Finally, the invention is directed to kit comprising the biochemical network allowing the uBCAAs detection and process for the preparation of said biochemical networks as diagnostic biomarker.

The present invention is directed to method of determining whether a subject is at risk of developing insulin resistance, particularly for advance alert of T2D onset in obese and non-obese subject, by detecting the branched-chain amino acids (BCAAs) present in an urine sample (uBCAAs) of the subjects. The present invention also relates to a method for determining the need of a dietary/nutritional supplement for a subject involving said uBCAAs biomarkers. Finally, the invention is directed to kit comprising the biochemical network allowing the uBCAAs detection and process for the preparation of said biochemical networks as diagnostic biomarker.

Non-communicable diseases (NCDs) are chronic diseases among which cardiovascular diseases (CVD) and Type 2 diabetes (T2D). While the worldwide prevalence of T2D still increases, CVD is the leading cause of death in the world. T2D and obesity are simultaneously manifestations and drivers of the CVD pathophysiology and altogether these NCD form the so-called cardiometabolic-based chronic disease (CMBCD). Insulin resistance (IR) is the common point between abnormal dysglycemia and adiposity impelling the progression of CMBCD.

Earlier identification of IR states as modifiable risk factor may improve the patients' health outcome and the social costs of CMBCD. However, wide population IR screening and monitoring through laboratory blood assays (e.g. HOMA and QUICKI indices), or euglycaemic hyperinsulinaemic (EH) clamp is uneasy. The assessment of IR using non-invasive and simplified technology is expected to facilitate mass screening campaigns.

Branched-chain amino acids (BCAA, i.e. Leucine, Isoleucine and Valine) have been implicated in IR genesis. Actually, BCAA play a central role on energy metabolism and the impairment of their catabolismmay culminate in hyperinsulinemia, lipogenesis and incomplete β-oxidation with accumulation of lipotoxic species (e.g. triacylglycerols (TAG) and diacylglycerols (DAG)). Moreover, impaired BCAA metabolism might stimulate the transport of free fat acids (FFA) through the endothelium in skeletal muscle with intracellular production of TAG and DAG culminating in peripheral IR 3-12

In the last decade, growing evidence of fasting blood branched-chain amino acids (bBCAA) as promising biomarkers for IR and future T2D was reported¹. The impairment of BCAA catabolismmay indeed contribute to the development of IR²⁻¹¹.

This is the object of the present invention.

Here, we describe the use of urinary BCAA as biomarkers of IR and as potential mass screening tool for advance alert of CMBCD risk. In our clinical study, blood and urine samples from normoglycaemic, normal weight/overweight and insulin sensitive/resistant subjects were evaluated. Compared to HOMA-IR index, the detection of uBCAA using our approach presented an accuracy of 88% for the diagnosis of IR. The easiness of collecting urine samples for the detection of high levels of uBCAA in large screening campaigns for IR would be beneficial for the early identification of patients at risk of T2D and CVD, hence allowing focused public health interventions for an effective prevention of these morbidities. We also present here a proof of concept of an engineered simplified rapid IR test for the detection of uBCAA employing DNA free synthetic biology principles, which provide a colorimetric readout in response to an uBCAA threshold, particularly by using a synthetic biochemical network to detect uBCAA, using particularly a synthetic biochemical network to detect uBCAA.

Here, we propose an innovative approach based on a rapid and simple detection of urine Branched-Chain Amino Acids to address the lack of easily usable tools for IR disclosure.

The methodology relies on in silico design and accurate system modelling and simulation, as well as experimental production using for example a robust microfluidic process.

They provide experimental evidence demonstrating the technological validity, and the advantages and efficiency of this novel diagnostic approach, particularly in clinical samples for the diagnosis of human pathologies.

In a first aspect, the present invention is directed to an in vitro method:
for determining whether a subject is at risk of being developing or to develop insulin resistance, and/or future T2D, CVD and/or associated pathologies; and/or
for advance alert of T2D, CVD and/or associated pathologies onset; and/or
for the detection of insulin-resistant subjects at risk of early T2D, CVD and/or associated pathologies onset; and/or
for the diagnostic and monitoring of insulin-resistance individual from an insulin-sensitive one or, the method comprising:
   a) from an urine sample obtained from the subject,
measuring/determining the concentration of branched-chain amino acids (BCAAs) present in said urine sample (uBCCAs) by an enzymatic reaction network by gathering the urine sample with a solution containing at least Leucine dehydrogenase (LeuDH) enzyme;
and
determining whether the subject is at said risk, wherein a uBCCAs concentration superior or egal to a cut-off (threshold)), is indicative that the subject is at risk of developing or develop insulin resistance, and/or future T2D, CVD and/or associated pathologies/for advance alert of T2D, CVD and/or associated pathologies onset/for the detection of insulin-resistant subjects at risk of early T2D, CVD and/or associated pathologies onset/can distinguish insulin-resistant individuals from insulin-sensitive one.

Preferably, the subject to be tested is a normoglycaemic subject, more preferably the glucose level of the subject is inferior to 7 mM in serum; or normoglycuric subject, more preferably the glucose level of the subject is inferior to 200 μM in urine.

In another aspect, the present invention relates to a method or a process of determining the need or deficiency of a dietary/nutritional supplement for a subject or to analyze the nutritional needs or deficiency of a subject by considering a combination of various health and performance factors (health profile);
comprising:
   a) from an urine sample obtained from the subject,
   measuring/determining at least the concentration of branched-chain amino acids (BCAAs) present in said urine sample (uBCCAs) by an enzymatic reaction network by gathering the urine sample with a solution containing at least Leucine dehydrogenase (LeuDH) enzyme;
   b) comparing the result obtained in step a) to the concentration of uBCAAs present in a subject exhibiting a normal health profile and/or control health profile(s) known to require a personalized regimen;
   c) determining whether the subject is in need or deficiency of nutritional/dietary supplement(s) and/or requires a personalized regimen or therapy wherein the presence of a uBCCAs concentration superior or egal to a cut-off (threshold)), is indicative that the subject is in need or deficiency of dietary/nutritional supplement(s) and/or personalized regimen or therapy;
   d) optionally,
   preparing a personalized regimen for the subject, the regimen including a customized nutritional formula and/or synergistic physical program and/or personalized therapy; and
   administering the regimen to the subject and/or performing the synergistic physical program and/or the personalized therapy over a period of time;
   e) optionally, further comprising re-analysing the subject's needs or deficiencies and, if necessary, adjusting the regimen or the therapy.

Preferably, the subject to be tested is a normoglycaemic subject, more preferably the glucose level of the subject is inferior to 7 mM in serum; or normoglycuric subject, more preferably the glucose level of the subject is inferior to 200 μM in urine.

Preferably the subject presents a pathology associated to an increase of BCAA, preferably selected from the group of abnormal renal function, non-alcoholic steatohepatitis (NASH), hypertension or cardiometabolic disorders pathologies.

This method for determining the need or deficiency of a dietary/nutritional supplement for a subject or to analyse their nutritional needs or deficiencies by considering a combination of various health and physical performance factors (health profile) is based on the subject's individual uBCCAs concentration. Based on this analysis, a personalized regimen can be formulated for the subject, wherein the regimen may include a broad range of nutrients and/or physical programs. The nutrients are used by the subject according to the regimen so as to improve or restore the subject to optimal health over a period of time. At periodic times during the supplementation or restriction, the subject's needs may be re-assessed and, if necessary, the regimen may be adjusted.

In a preferred embodiment of the method of the present invention, said the uBCAAs cut-off (threshold) is between 65 μM and 95 μM, preferably is between 70 μM and 90 μM, between 75 μM and 85 μM, more preferably 80 μM.

In a preferred embodiment of the method of the present invention, said uBCAAs cut-off used to determine the risk for the subject, or for determining the need of a dietary/nutritional supplement for a subject or to analyse their nutritional needs is the same for a subject obese or not.

In a preferred embodiment of the method of the present invention, in step a), the measure/determination of the concentration of uBCAAs is carried out by a method comprising the steps of:
a1) bringing into contact said urine sample with a solution containing Leucine dehydrogenase (LeuDH), β-Nicotinamide adenine dinucleotide hydrate (NAD+) and Thiazolyl Blue Tetrazolium Bromide (MTT);
a2) incubating the composition obtained in step a1);
a3) measuring the output signal generated at step a2); and
a4) determining from said output signal the concentration of uBCCAs.

In another preferred embodiment of the method of the present invention, in step a), the measure/determination of the concentration of uBCAAs is carried out by a method comprising in step a), a preliminary step wherein the urine sample of the subject is pre-incubated with ascorbate oxidase in order to eliminate the ascorbic acid, preferably in presence of 1-Methoxy-5-methylphenazinium methyl sulfate (1M-PMS), more preferably at 0.04 mM±0.0.02 mM 1M-PMS

In another preferred embodiment of the method of the present invention, in step a), the LeuDH and ascorbate oxidase enzyme are in pH 7.5 to pH 9 buffer solutions, preferably between pH 7.8 and pH 8.2, more preferably in pH 8 in for instance 3-(N-morpholino) propanesulfonic acid (MOPS) buffer, preferably in 200 mM MOPS buffer at pH 8.0

Others buffers having at a pH comprised between 7.5 and pH 9 can be used, preferably between pH 7.8 and pH 8.2, more preferably at pH 8. For example, but non limited to, buffer selected from the group consisting of 100 mM Tris HCl; the pair 100 mM MOPS, 200 mM CAPS; the pair 100 mM MOPS, 200 mM CAPSO; the pair 100 mM MOPS, 200 mM CHES; the pair 100 mM Citrate, 200 mM CAPS; the pair 100 mM Citrate, 200 mM CAPSO; the pair 100 mM Citrate, 200 mM CHES; the pair 100 mM MES, 200 mM CAPS; the pair 100 mM MES, 200 mM CAPSO or the pair 100 mM MES, 200 mM CHES.

In another preferred embodiment, in step a), the pre-incubation and/or the incubation is/are carried out at a temperature comprised between 20° C. and 60° C., more preferably comprised between 30° C. and 40° C., 37° C.±2° C. and 37° C. being the most preferred.

In another preferred embodiment of the method of the present invention, said in step a1), the LeuDH enzyme is selecting from the group consisting of Bacillus cereus LeuDH, preferably the Uniprot POA393 LeuDH, more, preferably Uniprot POA393-1), Bacillus stearothermophillus LeuDH, preferably the Uniprot P13154 LeuDH, Bacillus cereus LeuDH linked to a SUMO protein group and Bacillus stearothermophillus LeuDH linked to a SUMO protein group, Bacillus stearothermophillus optionally linked to a SUMO group being preferred.

In another aspect of the present invention, method comprises a step b) of determining the concentration of glucose present in said sample, said glucose determination being preferably carried out by an enzyme reaction in presence of glucose oxidase (GO), preferably GO and horse radish peroxidase (HRP) enzyme and Amplex red, more preferably in MOPS buffer, preferably in order to control the fasting of the subject.

In a preferred embodiment of the methods of the present invention, in step a), the measure/determination of the concentration of uBCAAs) and optionally the measure of concentration of glucose if performed are carried out on a sample from a fasted subject.

Are also preferred, the methods of the present invention wherein:
in step a) and in step b), if performed, the sample for each step is an urine sample from the subject; or
in step a) the sample is urine sample and in step b) the sample is a blood sample from the subject.

Are also preferred, the methods of the present invention wherein:
in step a) and, if performed, in step b), the samples are urine samples and the measure/determination of the concentration of uBCAAs and the glucose are carried out on two distinct samples from the subject.

In a more preferred embodiment of the methods of the present invention:
in step a) the solution containing at least LeuDH enzyme (uBCAAs biochemical network) is encapsulated in a vesicle system, preferably encapsulated within a liposome, a droplet, a polymeric support with selective permeability such as, but not limited to, polypeptides, PEG (polyethylene glycol), more preferably within bilipidic membrane vesicles or unilamellar membrane vesicles, more preferably within giant unilamellar vesicles (GUV), within small, unilamellar vesicles or Sonicated Unilamellar Vesicles” (SUV) or within large unilamellar Vesicles (LUV).

Methods for the preparation of GUV, SUV and LUV vesicles are wellknown by the person skilled in the art¹⁹.

For example, but not limited to, SUV can be prepared by sonication using a cup horn, bath, or probe tip sonicator. LUV can be prepared by a variety of methods including extrusion techniques, detergent dialysis (i.e. Di-Octylglucoside Vesicles), fusion of SUV), reverse evaporation or ethanol injection. Unilamellar vesicles can be prepared from multilamellar vesicles (MLV or from Large, Multilamellar Vesicles (LMV)). SUV are typically 15-30 nm in diameter while LUV range from 100-200 nm or larger GUV can be prepared by mixing different populations of SUVs.

In a preferred embodiment, in step b) if performed, the solution containing at least the glucose oxidase enzyme (glucose biochemical network) is encapsulated in a vesicle system, preferably encapsulated within a liposome, a droplet, a polymeric support with selective permeability such as, but not limited to, polypeptides, PEG (polyethylene glycol), more preferably within bilipidic membrane vesicles or unilamellar membrane vesicles, more preferably within GUV, within SUV or within LUV, preferably within the same vesicle system as for LeuDH enzyme biochemical network.

In a preferred embodiment, the vesicle system is produced by microfluidic process.

In a preferred embodiment, the vesicles are giant unilamellar vesicle (GUV) produced by microfluidic process, preferably by using the process named SKC3.1, said process SKC3.1 comprising the steps of:
a) After etching, silicon wafers are coated with a photoresistant layer (50 μm) and baked. Photolithography performed at 375 nm removes the unexposed resist to reveal microstructures:
b) Soft lithography of microfluidic chips was performed using polydimethylsiloxane (PDMS) to produce to microfluidic chip. PDMS microfluidic devices were treated with PVA and the GUV production was based on octanol-assisted liposome assembly (OLA);
c) The formation of vesicles on-chip was controlled via a pressure-driven pump by which flow rates of all three phases (inner aqueous (IA), intermediary lipid-octanol oil (LO) and outer aqueous (OA)) were tuned in real-time,
The first emulsion (water-in-oil; W/O) was generated in the first flow-focusing motif where LO wets the hydrophobic PDMS walls and surrounds IA phase, forming spontaneously an internal IA stream and a thin layer of LO phase between PDMS and IA:
In the second flow-focusing region, the OA phase is pumped in a high pressure provoking a shear stress and the pinch-off of the first emulsion W/O, these steps resulting in a double emulsion (water-in-oil-in-water; W/O/W), wherein:
phospholipids present in the LO phase spontaneously assemble along both water interfaces while the octanol-1 pockets are extracted to form GUVs, and
flows were in the range of 0.5-3 μL/min for IA, 0.2-2 μL/min for LO and 10-120 μL/min for OA. OA to IA flow ratio allows to set the size of the produced GUV (15-75 μm diameter) and the frequency of the production (3 000-10 000 Hz).

In a preferred embodiment, the GUV are obtained by the SKC3.1 process as described in Example 5,

Are also preferred, the methods of the present invention wherein:
in step a) and b) the vesicle containing the LeuDH biochemical network and the vesicle containing the glucose oxidase biochemical network are supported or entrapped on/into polymeric matrices as for instance hydrated alginate gels, chemical polymers, biodegradable polymers, rehydratable polymers, delivery polymeric systems and solid or semi-solid or sprayable matrices.

In another aspect, the present invention is directed to a kit comprising:
Ascorbate oxidase and LeuDH, preferably in solution in buffer as described above, preferably in MOPS; and optionally
Glucose oxidase and HRP, preferably in solution in buffer as described above, preferably in MOPS.

In a preferred embodiment, said kit comprises:
Ascorbate oxidase and LeuDH in solution in buffer as described above, preferably in MOPS; and
Glucose oxidase and HRP in solution in buffer as described above, preferably in MOPS.

In another preferred embodiment, said kit comprises:
Ascorbate oxidase and LeuDH, preferably in solution in buffer as described above, preferably in MOPS, encapsulated in a vesicle system, preferably in GUV, SUV or LUV vesicles, preferably GUV obtained by microfluidic process;
and optionally
Glucose oxidase and HRP, preferably in solution in buffer as described above, preferably in MOPS, encapsulated in a vesicle system, preferably in GUV, SUV or LUV vesicles, preferably GUV obtained by microfluidic process.

In another preferred embodiment, said kit comprises:
Ascorbate oxidase and LeuDH, preferably in solution in buffer as described above, preferably in MOPS encapsulated in a vesicle system, preferably in GUV, SUV or LUV vesicles, preferably GUV obtained by microfluidic process; and
Glucose oxidase and HRP in solution in buffer as described above, preferably in MOPS, encapsulated in a vesicle system, preferably in GUV, SUV or LUV vesicles, preferably GUV obtained by microfluidic process.

In another aspect of the present invention, said kit comprises beads, preferably alginate beads, wherein the beads contain or entrap:
Ascorbate oxidase and LeuDH, preferably in solution in buffer as described above, preferably in MOPS encapsulated in a vesicle system, preferably in GUV, SUV or LUV vesicles, preferably GUV obtained by microfluidic process; and optionally
Glucose oxidase and HRP in solution in MOPS encapsulated in a vesicle system, preferably in GUV, SUV or LUV vesicles, preferably GUV obtained by microfluidic process.

In a preferred embodiment, said kit comprises beads, preferably alginate beads, wherein the beads contain or entrap:
Ascorbate oxidase and LeuDH in solution in buffer as described above, preferably in MOPS encapsulated in a vesicle system, preferably in GUV, SUV or LUV vesicles, preferably GUV obtained by microfluidic; and
Glucose oxidase and HRP in solution in MOPS encapsulated in a vesicle system, preferably in GUV, SUV or LUV vesicles, preferably GUV obtained by microfluidic process.

In a preferred embodiment, in said kit of the present invention, the GUV vesicles are obtained by the process named SKC3.1 comprising the steps of:
a) After etching, silicon wafers are coated with a photoresistant layer (50 μm) and baked. Photolithography performed at 375 nm removes the unexposed resist to reveal microstructures:
b) soft lithography of microfluidic chips was performed using polydimethylsiloxane (PDMS) to produce to microfluidic chip. PDMS microfluidic devices were treated with PVA and the GUV production was based on octanol-assisted liposome assembly (OLA);
c) the formation of vesicles on-chip was controlled via a pressure-driven pump by which flow rates of all three phases (inner aqueous (IA), intermediary lipid-octanol oil (LO) and outer aqueous (OA)) were tuned in real-time,
the first emulsion (water-in-oil; W/O) was generated in the first flow-focusing motif where LO wets the hydrophobic PDMS walls and surrounds IA phase, forming spontaneously an internal IA stream and a thin layer of LO phase between PDMS and IA:
in the second flow-focusing region, the OA phase is pumped in a high pressure provoking a shear stress and the pinch-off of the first emulsion W/O, these steps resulting in a double emulsion (water-in-oil-in-water; W/O/W), wherein:
phospholipids present in the LO phase spontaneously assemble along both water interfaces while the octanol-1 pockets are extracted to form GUVs, and
flows were in the range of 0.5-3 μL/min for IA, 0.2-2 μL/min for LO and 10-120 μL/min for OA. OA to IA flow ratio allows to set the size of the produced GUV (15-75 μm diameter) and the frequency of the production (3 000-10 000 Hz).

In a preferred embodiment, the GUV are obtained by the SKC3.1 process as described in Example 5;

Finally, the present invention is directed to a method for the production of GUV vesicles, said method comprising the steps of:
a) After etching, silicon wafers are coated with a photoresistant layer (50 μm) and baked. Photolithography performed at 375 nm removes the unexposed resist to reveal microstructures:
b) soft lithography of microfluidic chips was performed using polydimethylsiloxane (PDMS) to produce to microfluidic chip. PDMS microfluidic devices were treated with PVA and the GUV production was based on octanol-assisted liposome assembly (OLA);
c) the formation of vesicles on-chip was controlled via a pressure-driven pump by which flow rates of all three phases (inner aqueous (IA), intermediary lipid-octanol oil (LO) and outer aqueous (OA)) were tuned in real-time,
the first emulsion (water-in-oil; W/O) was generated in the first flow-focusing motif where LO wets the hydrophobic PDMS walls and surrounds IA phase, forming spontaneously an internal IA stream and a thin layer of LO phase between PDMS and IA:
in the second flow-focusing region, the OA phase is pumped in a high pressure provoking a shear stress and the pinch-off of the first emulsion W/O;
these steps resulting in a double emulsion (water-in-oil-in-water; W/O/W), wherein:
phospholipids present in the LO phase spontaneously assemble along both water interfaces while the octanol-1 pockets are extracted to form GUVs.
flows were in the range of 0.5-3 μL/min for IA, 0.2-2 μL/min for LO and 10-120 μL/min for OA. OA to IA flow ratio allows to set the size of the produced GUV (15-75 μm diameter) and the frequency of the production (3 000-10 000 Hz).

In a preferred embodiment, the invention is directed to the method for the production of GUV as described in Example 5.

The present invention is also directed to the use of the GUV obtained by the process SKC3.1 as describe above according to the present invention, preferably the use of the process SKC3.1 as described in Example 5 for encapsulated biochemical network, preferably the biochemical network allowing determining whether a subject is at risk of being developing or to develop insulin resistance/future T2D and/or CVD/for advance alert of T2D and/or CVD onset in a patient from an urine sample, as described in the present invention.

The following examples, the figures and the legends hereinafter have been chosen to provide those skilled in the art with a complete description to be able to implement and use the present invention. These examples are not intended to limit the scope of what the inventor considers to be its invention, nor are they intended to show that only the experiments hereinafter were carried out.

Other characteristics and advantages of the invention will emerge in the remainder of the description with the Examples and Figures, for which the legends are given hereinbelow.

Figures legends:

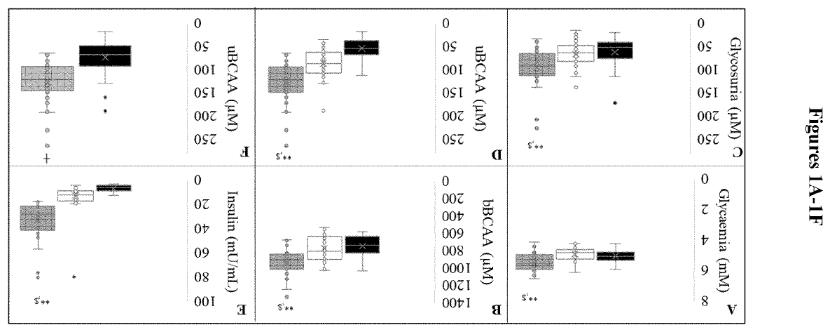

FIG. 1: Blood and urine biochemical parameters compared in the three groups. (A) Glycaemia, (B) bBCAA, (C) Insulin, (D) Glycosuria and (E) uBCAA. bBCAA and uBCAA quantification presented here were performed using the SKC synthetic biochemical network method. Black boxes are NWIS; white are for OWIS and grey ones for OWIR. (F) The black box represents the gathered composite insulin-sensitive individuals (CIS-HOMA≤4) and the grey box OWIR. Comparison between groups were performed with Student (p<0.05 for *NWIS vs OWIS; **NWIS vs OWIR; ^{S}OWIS vs OWIR and ^{†}CIS vs OWIR). NWIS (Normal-weight insulin-sensitive); OWIS (Overweight insulin-sensitive); OWIR (overweight insulin-resistant).

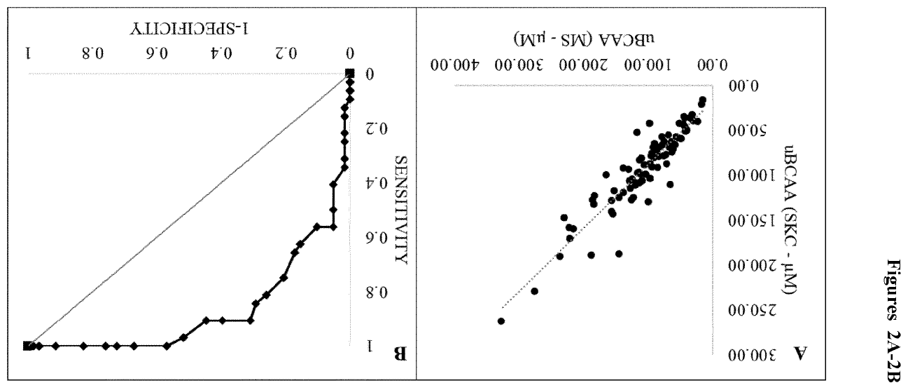

FIGS. 2A-2B: Analytical validation and evaluation of diagnostic performance of uBCAA versus HOMA-IR index. (A) The SKC synthetic biochemical network method for the quantification of uBCAA was validated by the reference standard method (LC-MS/MS). Intraclass Correlation Coefficient between the two methods was 0.87 (r²=0.86). (B) ROC regression for the diagnostic performances of SKC quantification of uBCAA evaluated against HOMA-IR (cut-off>4.0) as reference test (AUC=0.88).

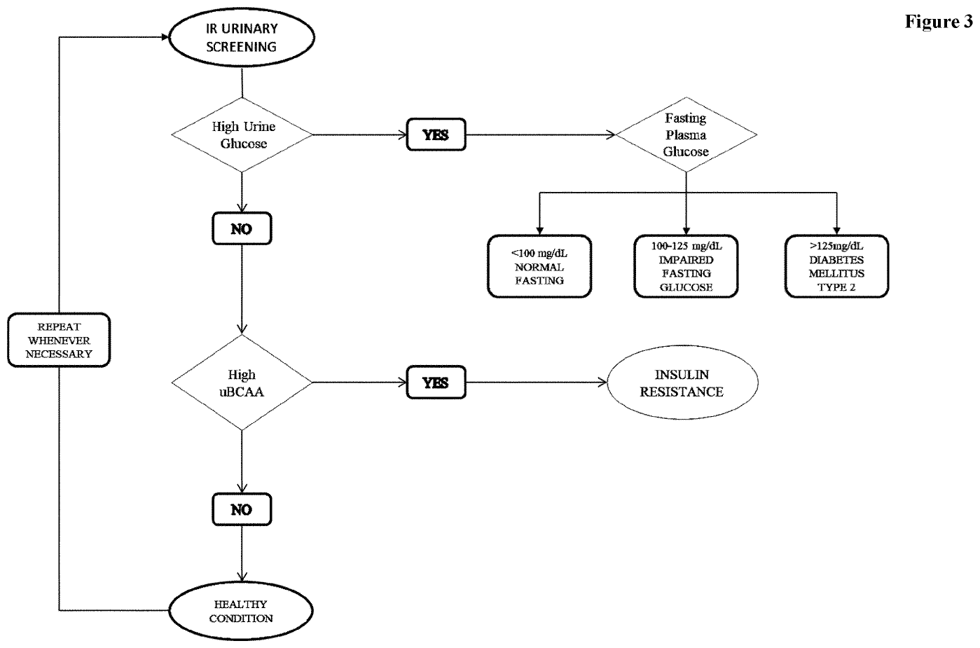

FIG. 3: Insulin Resistance Urine Test Algorithm. The proof of concept we developed are based on urine BCAA and Glucose detection. Subjects with high urinary BCAA levels are diagnosed as holding an insulin resistant status. Subjects with high uBCAA levels presenting high glycosuria are candidates for possible T2D and should perform a fasting blood glucose measurement for confirmation of T2D.

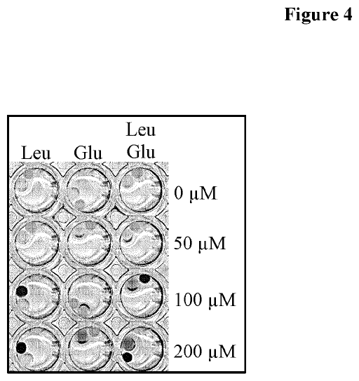

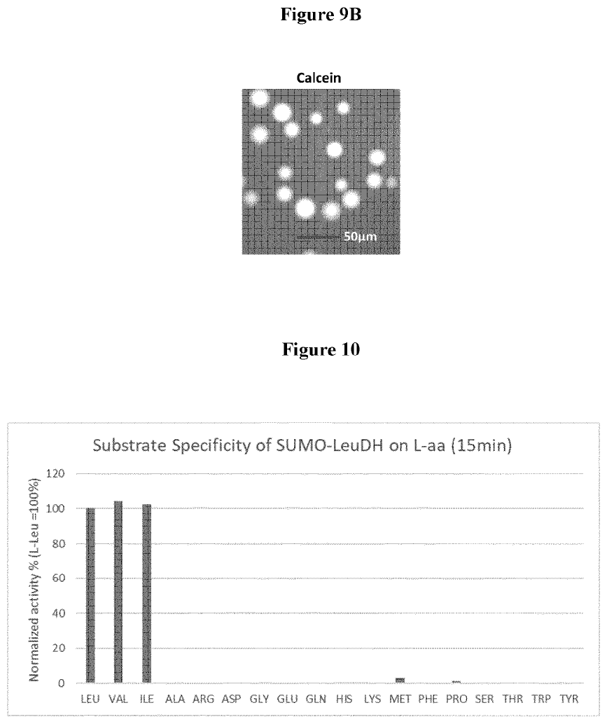

FIG. 4: Innovative approach for IR detection. The detection of IR is performed according to the medical algorithm proposed using glycosuria and uBCAA detection. SKC synthetic biochemical networks containing enzymes for the detection of either BCAA or Glucose are encapsulated into Giant Unilamelar Vesicles (GUV). GUVs were prepared using a microfluidic setup as described in the Methods Section. For visualising purposes, GUVs were produced using a fluorescent phospholipid bilayer membrane (DPPC:DOPC:CHO (4.5; 4, 5; 1), DiIC18 (0.5 mol %) (see FIG. 9A) and encapsulating a 1 μM calcein solution (see FIG. 9B) into the internet aqueous phase. Red scale bars represent 50 μm. (Right panel) GUVS containing the synthetic biochemical networks for the detection of either BCAA or Glucose were entrapped into alginate beads. Solutions containing BCAA (Column B), glucose (G) or both together (B+G) were incubated with at different concentrations with BCAA and Glucose-Alginate beads. BCAA-Alginate beads (blue) and Glucose-alginate beads (violet) respond specifically to BCAA and glucose, respectively, when the concentrations of the substrates are superior to the threshold of 100 μM.

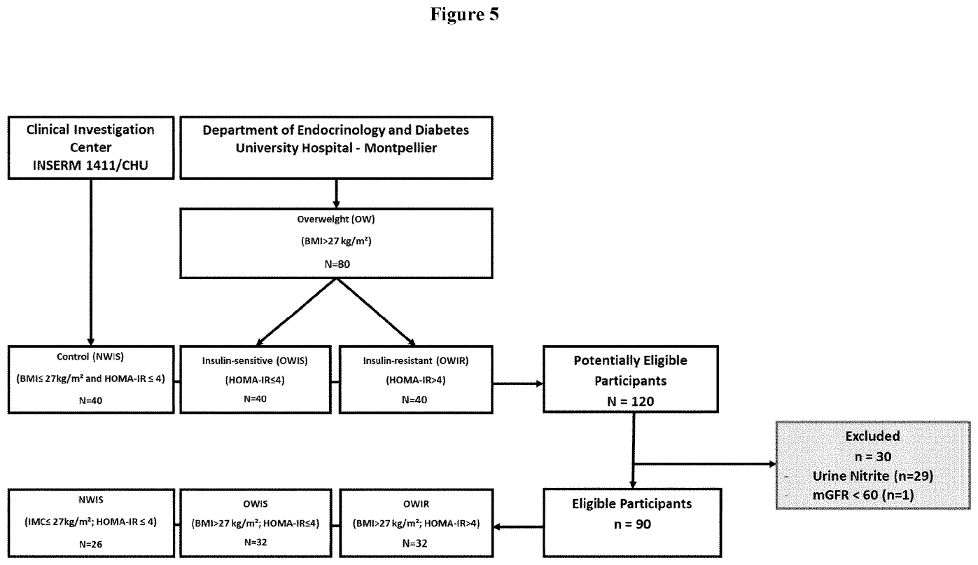

FIG. 5: Study flow diagram. INSERM, National Institute of Health and Medical Research; CHU, University Hospital Center; OW, Over Weight; BMI, Body Mass Index; HOMA-IR, Homeostasic Model Assessment of Insulin Resistance; NWIS, Normal Weight Insulin-Sensitive; OWIS, Over Weight Insulin-Sensitive; OWIR, Over Weight Insulin-Resistant; eGFR, estimated Glomerular Filtration Rate.

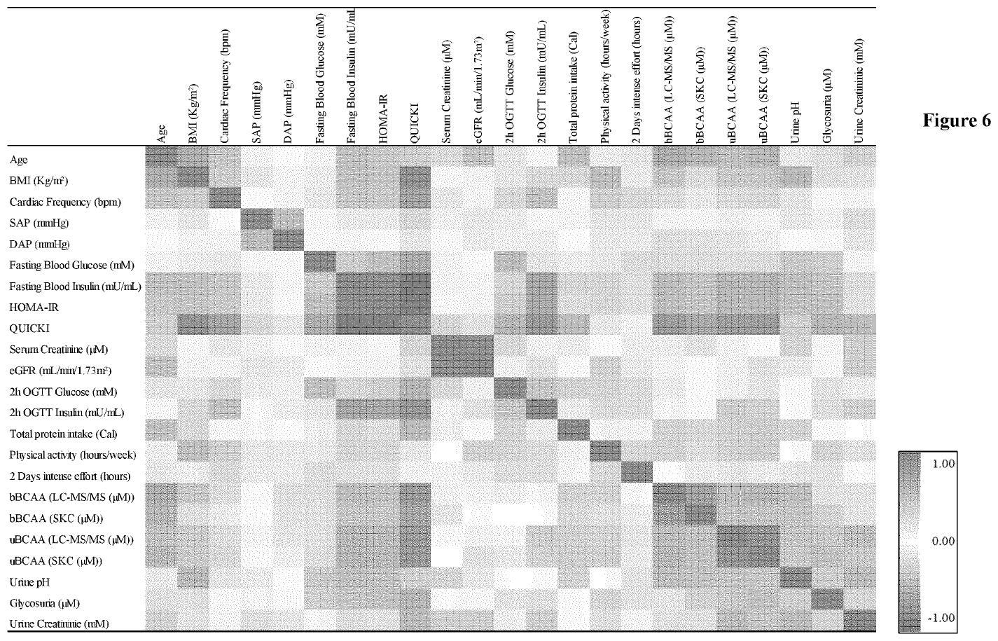

FIG. 6: Association of demographic, clinical and biochemical data. Correlations were evaluated using Pearson correlation coefficient. Colours are proportional to the strength of associations. BMI (Body Mass Index); SAP (Systolic Arterial Pressure); DAP (Diastolic Arterial Pressure); eGFR (estimated Glomerular Filtration Rate) OGTT (Oral Glucose Tolerance Test); bBCAA (blood Branched-Chain Amino acids); uBCAA (urine Branched-Chain Amino acids); and SKC (Synthetic Biochemical Network for BCAA detection).

FIGS. 7A-7B: Linear regression between Urine Creatinine and uBCAA. (A) Linear regression was analysed by group or using the entire cohort (B) ( [symbol] ) Blue dots are NWIS, ( [symbol] ) are OWIS and ( [symbol] ) grey dots are OWIR. ( [symbol] ) Violet dots represent the entire cohort. Subjects presenting nitrite in urine samples and eGFR<60 mL/min/1.73 m² were included in this analysis (n=120). uBCAA were quantified using the SKC synthetic biochemical approach.

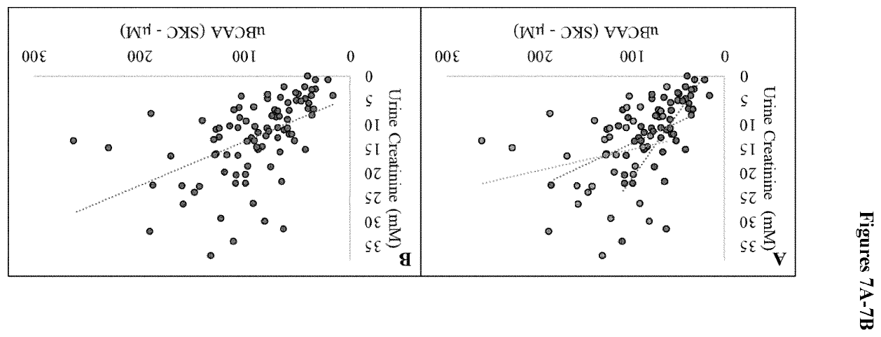

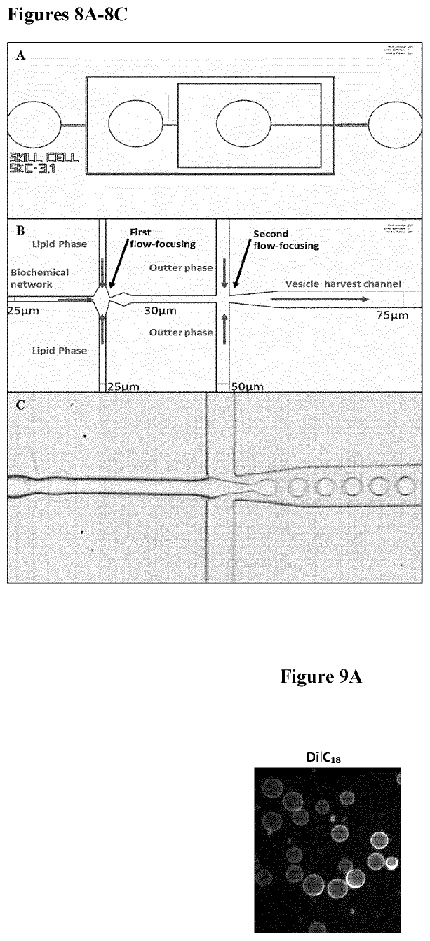

FIGS. 8A-8C: Microfluidic device used for Giant Unilamellar Vesicle production. The homemade microfluidic device (A) uses two consecutive flow-focusing (B) for the production of the double-emulsions. Giant unilamelar vesicles production (C) containing the biochemical networks used for BCAA and glucose detection.

FIGS. 9A-9B: The production process using the microfluidic double emulsion device with 1 μL/min at the IA (inner aqueous solution), 15 μL/min at the OA (outer aqueous solution) and 0.5 μL/min at the LO (lipid oil). Fluorescence images of GUVs with lipid composition DPPC:DOPC:CHO (4.5; 4, 5; 1), DiIC18 (0.5 mol %) (A) containing calcein in IA phase (1 μM (B).

FIG. 10: Oxidation of L-amino acids at 15 minutes at 37° C. Oxidation of L-amino acids was followed by colorimetric reduction of Thiazolyl Blue Tetrazolium Bromide. Bars show normalized values to the oxidation of L-Leucine (100%). Convention three letters abbreviations were used to refer to L-amino acids.

### EXAMPLE 1: MATERIALS AND METHODS

### A) Materials

1,2-Dioleoyl-sn-glycero-3-phosphocholine (DOPC) and 1,2-dipalmitoylphosphatidylcholine (DPPC) were purchased from Avanti Polar Lipids Inc. MTT (3-[4, 5-dimethylthiazol-2-yl]-2, 5-diphenyltetrazolium bromide) and AmplexRed® (10-Acetyl-3,7-dihydroxyphenoxazine) were purchased from Thermofisher. Nicotinamide adenine dinucleotide (NAD+), 1-methoxy-5-methylphenazinium methyl sulfate (1-methoxyPMS), L-Leucine, D-Glucose, Ascorbate oxidase (Cucurbita spp.), Glucose oxidase (Aspergillus niger), Peroxidase from horseradish (HRP), cholesterol, alginic acid sodium salt (medium viscosity), calcium chloride, sodium chloride, sulfuric acid, hydrogene peroxide (30% v/v), Poly(vinyl alcohol) (average mol wt 30,000-70,000) and Poloxamer188® (pluronic F68) were purchased from Sigma-Aldrich. Leucine dehydrogenase (Bacillus stearothermophilus) was from Creative Enzymes (NATE-1905). Polydimethylsiloxane (PDMS) and curing agent (Kit Sylgard 184) were obtained from Dow silicones (DOW EUROPE GMBH). Silicon wafers (ID-452) were obtained from UniversityWafer Inc. SU-8 negative photoresist (3050) and the development solution were purchased from Chimie Tech services (CSI). All other chemicals were of analytic grade quality.

### B) Human Samples Collection

From the 330 participants to be recruited at the end of this clinical study, 110 subjects will be included in the control group and 220 will be equally distributed in OWIS and OWIR groups. An interim analysis, presented here, was planned including 120 participants distributed equally among the three groups (FIG. 5).

The number of subjects recruited was estimated using a statistical power of 80% (risk of first species alpha of 0.05 and risk of second species beta of 0.20, using a T-Student Test). We considered a size effect of 38% in BCAA levels between insulin resistant and insulin sensitive subjects base on the previous studies^{5,46,47}. A lost rate range of 5-10% of samples was taken into account in our calculation.

### C) Statistical Analysis

Values were expressed as mean+/−standard deviation (SD) for continuous variables and number with percentages for categorical ones. Comparisons of the clinical groups were performed with Student test for continuous variables. Pearson correlation coefficient and Intraclass Correlation Coefficient were used for correlation and concordance analysis.

The performance of SKC method for uBCAA quantification as diagnostic tests was evaluated by their sensitivity and specificity. Sensitivity was the proportion of positive index test (defined as uBCAA concentration above the cut-off) in the insulin resistant population and specificity the proportion of negative (defined as uBCAA concentration under the cut-off) in the insulin sensitive population, according to the HOMA-IR index (HOMA-IR>4.0 for IR). Accuracy was also reported as the area under the curve using a ROC regression.

### D) Sample Pre-Treatment

After collection, blood and urine samples were kept at 4° C. until analysis, pre-treatment or freezing (−80° C.). At Sys2Diag, crude urine samples were analysed using urine strips for pH (Lyphan, R4979), nitrite (Quantofix Nitrites, Macherey-Nagel 91338), creatinine and albumin (Microalbustix, Siemens 04960872) before pre-treatment. Then, the samples were transferred to 50 ml sterile PP tubes for centrifugation 4200×g for 10 minutes at 4° C. Then, centrifuged urines were sequentially filtered (0.45 and 0.22 μm) and samples of 1.5 mL were frozen (−80° C.) until analysis. Alternatively, non-centrifuged or non-filtered urine samples were also stored at −80° C. in order to compare untreated versus treated urine.

Blood samples were collected in dry tubes. Tubes were centrifuged at 2 000 g for 10 minutes, at 4° C. in order to obtain serum. Serum samples (200 μL) were stored at −80° C. in PP tubes until analysis.

For all samples, the time between collection and freezing did not exceed 3 hours.

### E) Urine Creatinine Quantification

Creatinine concentrations in urine samples were also determined using the Creatinine Assay Kit from Sigma Aldrich (MAK080).

Briefly, pre-treated samples were thawed on ice. An additional centrifugation was performed in order to remove precipitates (21 000 g, 10 min at 4° C.). Following a dilution of urine samples (1:100-v:v) in creatinine assay buffer, 25 L of this mix was incubated with 25 μL of the Test solution or Control solution for 60 min at 37° C. in a microplate (Greiner Bio-One, Half Area Plate, #675101). The reaction was monitored at 570 nm with absorbance measurement every minute for 1 hour. Creatinine concentration was determined using a standard curve after normalization of each sample to its own control.

### F) SKC Synthetic Biochemical Network for BCAA Quantification

The assay is based on the selective oxidation of BCAAs by the enzyme Leucine dehydrogenase generating a colored indicator, MTT formazan. Briefly, three solutions were prepared for the assay.
BCAA A Solution (BAS) containing NAD+50 mM, 1M-PMS 200 μM and Ascorbate Oxidase 10 U/mL;
BCAA B Test Solution (BBTS) containing Leucine Dehydrogenase 75 U/mL and MTT 2 mM;
BCAA B Control Solution (BBCS) containing only MTT 2 mM.

All solutions were prepared in MOPS 200 mM, pH 8.0 buffer.

For urinary BCCA (uBCAA) quantification, pre-treated unfrozen samples were diluted 1:1 (v:v) in the MOPS buffer (200 mM, pH 8.0). For blood BCAA (bBCAA), serum samples were diluted 1:15 (v:v) in the same buffer.

Diluted samples (53 μL) were incubated with BAS (8 μL) for 5 min, 37° C. in a microplate. Then, either BBTS or BBCS (4 μL) was added and incubated for 30 min at 37° C. Kinetics of the reactions were monitored at 600 nm with absorbance measurements every minute.

Each sample was analysed with both BBTS and BBCS. BBCS allows to quantify the absorbance background generated by the residual ascorbic acid contained in samples and to normalize BCAA quantification with respect to its background. BCAA were quantified by comparing to the standard curve (200-12.5 μM) after subtraction of BBTS absorbance to that obtained with BBCS. All the tests were performed in duplicates and in two independent experiments.

Fresh untreated urine samples were also analysed following the same protocol and no differences were observed between treated and untreated urine samples (data not shown).

### G) SKC Synthetic Biochemical Network for Urine Glucose Quantification

Urine glucose quantification is based on the canonical oxidation of glucose by Glucose Oxidase (GO) coupled to Horseradish Peroxidase (HRP) with a final production of a colored indicator Resorufin.

Briefly, pre-treated unfrozen urine samples were diluted (1:1-v:v) in MOPS buffer (200 mM, pH 8.0). Previously diluted samples (50 μL) were prepared in two separated series in a microplate and incubated for 5 min at 37° C. Then, 50 μL of the Glucose Test Solution (GTS, containing 2 U/mL Glucose Oxidase, 0.4 U/mL HRP and 100 μM AmplexRed® in 200 mM MOPS buffer pH8.0) was added to one of samples' series. For the second samples' series, 50 μL of Glucose Control Solution (GCS, containing only 0.4 U/mL HRP and 100 μM AmplexRed® in the same buffer) was added. An additional incubation of 35 min at 37° C. was performed and the production of Resofurin was measured (570 nm).

Glucose was quantified by comparison with the standard curve (100-6.3 μM) after subtraction of GTS absorbance to that obtained with GCS. All the tests were performed in duplicates and in two independent experiments.

### H) LC-MS/MS

For sample preparation, 10 μL of urine or sera were treated with 1000 μL of 0.5 mM perfluoroheptanoic acid containing the internal standards. After mixing, 5 μL of sulfosalicylic acid was added and centrifugation was performed for 30 min at 6 000 g. The supernatants were transferred to the autosampler vials for analysis from which 10 μl of each sample was injected for LC-MS/MS analysis.

The LC-MS/MS analysis was performed on UPLC Acquity (Waters Corporation) coupled to a triple-quadrupole mass spectrometer XevoTQD (Waters Corporation).

Isoleucine, leucine and Valine were alaysed by a reversed-phase column (Acquity UPLC BEH C18, 2.1×10 mm, 1.8 μm, Waters Corporation). The chromatographic mobile phase was constituted of 0.5 mM perfluoroheptanoic acid in water (Phase A) and 0.5 mM perfluoroheptanoic acid in acetonitrile (Phase B) delivered at a flow rate of 0.65 mL/min at 40° C.

The gradient was 0-14 min, 99.5% to 70% A; 14-17.5 min, 70% A; 17.5-18.5 min, 70% to 99.5% A and 18.5-30 min 99.5% A to equilibrate the column again. The total run time was 30 min.

Isoleucine, leucine, valine, 2H3-leucine and 2H8-Valine ionization was performed using positive electrospray ionization of [M+H]+ and detected by multiple reaction monitoring. The source and capillary temperature were set to 150° C. and 650° C., respectively.

### I) Microfabrication of Microfluidic Device

Our microfluidic chip design is an optimization of a geometry described previously48 (FIGS. 8A-8B). Silicon wafers (100 mm) were etched with piranha solution (H₂SO₄:H₂O; 3:1). Spin-coating of SU-8 3050 resin was performed at 3 000 rpm for 30 seconds with a ramp of 300 rpm/s in a WS-650-23NPP spin coater (Laurell Technologies) to obtain a 50 μm photoresistant layer. Soft baking was performed at 80° C. for 45 minutes. Photolithographies were performed on the maskless system, Dilase-250 (KLOE) using the 375 nm laser (70 mW) with optimized power and writing speed. A post-exposure baking at 70° C. for 60 minutes was performed. Developing solution was used to remove the unexposed resist to reveal microstructures. A final hard baking step at 200° C. for 8 hour was done before use.

Soft lithography of microfluidic chips was performed using polydimethylsiloxane (PDMS) and its curing reagent (9:1 ratio). The mixture was degassed and poured onto the microstructure mold and then baked at 70° C. for 3 hours. All inlets and outlets holes were created using 1.5 mm biopsy punches. The PDMS chip was then treated with oxygen plasma (Corona SB, ElveFlow) for 2 minutes. Next, both the PDMS chip and a PMDS-coated glass slide were bonded together. Finally, the bonded chip was baked at 90° C. for 30 minutes and let cool down before use.

### J) Skillcell Design and Production

We previously developed a method to produce non-living vesicles for biomarkers detection through the implementation of programmable synthetic biochemical networks (Skillcells®)^{12-14,15}.

High-throughput preparation of monodispersed GUVs (Giant Unilamellar Vesicle) was achieved using our microfluidic chip design in which the GUV production was based on octanol-assisted liposome assembly (OLA).

Briefly, PDMS microfluidic devices were treated with PVA (Poly(vinyl alcohol); 1% w/v) to render hydrophilic the vesicle harvest channel (FIGS. 8A-8B). The formation of vesicles on-chip was controlled via a pressure-driven pump (MFCS EZ, Fluigent) by which flow rates of all three phases (inner aqueous (IA), intermediary lipid-octanol oil (LO) and outer aqueous (OA)) could be tuned and monitored in real-time. The corresponding microfluidic chip inlets and design are shown in FIGS. 84-8B.

For all experiments, LO phase stock solution consisted of 175 mM DOPC:DPPC:CHO (45:45:10) in ethanol and was stored at −20° C. in a nitrogen atmosphere. The LO stock solution was diluted (1:10, v:v) in 1-octanol in order to obtain the final concentration (17.5 mM) immediately before GUC production. The OA phase consisted in 10 mg/mL Poloxamer188® and 15% (v/v) glycerol in Milli-Q water. IA phases were customized according to the test (BCAA/Glucose) and corresponded to BBN (BCAA Biochemical Network, i.e. BAS+BBTS) and GTS, respectively added of 10 mg/mL Poloxamer188® and 10% (v/v) glycerol.

The first emulsion (water-in-oil; W/O) was generated using a flow-focusing design (FIGS. 8A-8B) where LO phase wets the hydrophobic PDMS walls and surrounds IA phase, forming spontaneously an internal IA stream and a thin layer of LO phase between PDMS and IA. In the second flow-focusing region, the OA phase is pumped in a high pressure provoking a shear stress and the pinch-off of the first emulsion W/O. It results in a double emulsion (water-in-oil-in-water; W/O/W). Phospholipids present in the LO phase spontaneously assemble along both water interfaces while the octanol-1 pockets are extracted to form GUVs. Flows were in the range of 0.5-3 μL/min for IA, 0.2-2 μL/min for LO and 10-120 μL/min for OA. OA to IA flow ratio allows to set the size of the produced GUV (15-75 μm diameter) and the frequency of the production (3 000-10 000 Hz).

GUV production was followed on a Leica microscope (DMIL) coupled to a high-speed camera (Phantom VEO410-L). The production frequency was calculated based on the recorded movies to count particles in a fixed period. Size measurements of Guvs were performed in a Malassez counting chamber using IMAGE-J software. Size and frequency were varied during experiments to evaluate the influence of production parameters on GUV production.

GUVs production by a process using the microfluidic double emulsion device is depicted in FIGS. 9A-9B with 1 μL/min at the IA (inner aqueous solution), 15 μL/min at the OA (outer aqueous solution) and 0.5 μL/min at the LO (lipid oil). See legends of these figures.

### K) Functional Alginate Hydrogel Production

GUV are constituted of an outer aqueous phase (OA). Our Skillcells® (non-living vesicles containing the programmable synthetic biochemical networks) can be used with several types of hydrogel to obtain functionalized macrocapsules. To form Skillcell-containing beads, GUVs containing either BCAA (BCAA-Alginate Bead) or Glucose (Glucose-Alginate Bead) detection biochemical network were mixed with an alginate solution to a final concentration of 1.4% (w/v). This mixture was extruded dropwise with a syringe into a CaCl₂) bath solution (50 mM) with gentle agitation for 5 minutes to cure alginate beads. The functional beads are harvested after precipitation in the bath followed of two washing steps with OA and Milli-Q water.

The optimal number of GUVs entrapped into the beads may be adjusted by simple dilution/concentration of the GUV solution before mixing with alginate. The syringe height and dropwise speed are also tuneable parameters to customise the bead's size in order to design the best format according to the concentration of biomarkers inside the matrices and samples.

The Skillcell-containing alginate macrobeads were tested in the presence of their respective biomarkers. BCAA and Glucose-Alginate Beads were set to produce a visible output only if the concentration of biomarkers was superior to 100 μM. This was achieved by varying the number of GUVs entrapped into alginate beads in order to obtain beads which are capable of responding to different thresholds of biomarkers (not shown).

BCAA-Alginate Beads were stored overnight in a Tris-HCl buffer (100 mM, pH7.8) containing MTT (0.4 mM) before testing. Similarly, Glucose-Alginate Beads were loaded in Tris-HCl buffer (100 mM, pH 7.8) containing AmplexRed® (1 mM).

Functionalized beads were incubated 15 minutes at 37° C. in the presence of L-Leucine, D-Glucose or both at different concentrations (0; 50; 100 and 200 μM) in Tris-HCl buffer (100 mM pH7.8). After 15 minutes, the colorimetric signal was recorded.

### EXAMPLE 2: CLINICAL STUDY DESIGN

### Clinical Study Design

Subjects whose data was used for the presented interim analysis (n=120; FIG. 5) were recruited between September 2019 and July 2021. Overweight non-diabetic adults coming to the Endocrinology and Diabetes Department of University Montpellier Hospital for routine medical consultation were prospectively invited to participate. Normal weight controls were invited to participate by the Clinical Investigation Centre of the same hospital.

Participants were divided into three groups according to their IR status (according to the reference HOMA-IR value) and BMI. Normal weight, insulin-sensitive (NWIS) persons presented simultaneously BMI≤27 kg/m² and HOMA-IR≤4.0; overweight insulin-sensitive (OWIS) persons BMI>27 kg/m² and HOMA-IR≤4.0; and overweight insulin-resistant (OWIR) persons BMI>27 kg/m² and HOMA-IR>4.0.

HOMA-IR index and BMI are calculated as follow:

$$HOMA - IR = ( Fasting insulin * Fasting glucose ( mM ) / 22.5 .$$

$$B M I = Weight ( kg ) / {Height}^{2} ( {m}^{2} ) .$$

Symptoms, risk factors, life habits, medical history, comorbidities and treatments were collected by the practitioner. Physical activity and nutrition were informed on a questionnaire. Fasting blood and fasting urine samples of participants were collected simultaneously. Blood was collected in two dry tubes (no additive) for sera preparation and urine (the second urination of the day) was collected in a 100 mL sterile polypropylene pot. Samples were immediately stored at 4° C. One tube of blood and the pot of urine for each participant were sent within 3 hours to the research laboratory Sys2Diag. Once at Sys2Diag, each sample was analysed by independent biologists blinded of the clinical status of the participants. Biochemical parameters measurements included urine pH, urine creatinine, glycosuria, uBCAA quantified by both liquid chromatography-tandem mass spectrometry (LC-MS/MS) and the synthetic biochemical network we developed (SKC). bBCAA was quantified by LC-MS/MS and SKC synthetic biochemical network as well. The second tube containing blood was analysed at University Hospital laboratory following the routine protocols.

A French ethical committee (CPP-Ile de France VII) approved the study and all subsequent amendments on Aug. 23, 2019. The study was registered at www.clinicaltrials.gov (NCT04010903). All methods were performed in accordance with relevant guidelines and regulations. All participants signed informed consent prior to participating.

Samples of 90 subjects were analysed from the total 120 individuals enrolled in this monocentric study. Among the thirty participants excluded from this analysis, twenty-nine had nitrite detected in urine (>0.0 mg/mL) indicating bacterial contamination and one subject had an estimated glomerular filtration rate (eGFR)<60 mL/min/1.73 m² meaning a kidney disease (FIG. 5).

### Demographics and Clinical Characteristics

Demographics and clinical data are shown in Table 1. The female to male sex ratio was 2.75 and the mean age was 50.8 years (SD±14.0). Concerning the entire cohort, cardiac frequency moderately correlates to BMI (r=0.46). Further, moderate associations were observed for fasting plasma insulin and (1) BMI (r=0.55), (2) fasting blood glucose (r=0.46) and (3) fasting urine glucose (r=0.52) (FIG. 6).

Despite OWIR were younger than OWIS and NWIS, no significant differences were observed between groups concerning neither blood pressure (systolic and diastolic arterial pressure-SPA and DAP, respectively), serum creatinine nor estimated glomerular filtration rate (eGFR) (Table 1A). Fasting blood glucose was higher in OWIR compared to both NWIS and OWIS but no difference was observed between NWIS and OWIS. A similar pattern was observed for fasting urine glucose (Table 1, FIGS. 1A and D). Although not reaching the thresholds for a qualification of IR, the group of OWIS had higher fasting plasma insulin, and higher HOMA-IR and QUICKI indices than NWIS. As expected, OWIR group presented the highest fasting plasma insulin, HOMA-IR and QUICKI indices compared to the two other groups (Table 1A and FIG. 1C).

TABLE 1A
Demographics and clinical characteristics of participants. Unless otherwise indicated, data are reported
as mean (S.D.). Missing data for some participants are indicated specifically for each variable. NWIS
(Normal-weight insulin-sensitive); OWIS (Overweight insulin-sensitive); OWIR (overweight insulin-resistant);
SAP (Systolic Arterial Pressure); DAP (Diastolic Arterial Pressure); OGTT (Oral Glucose Tolerance Test); eGFR
(estimated Glomerular Filtration Rate) and BMI (Body Mass Index). Comparison between groups were performed using
t-Student test (p < 0.05 for *NWIS vs OWIS; **NWIS vs OWIR and ^{$}OWIS vs OWIR).

| | NWIS N = 26 | | OWIS N = 32 | | OWIR N = 32 | | TOTAL N = 90 | |
|---|---|---|---|---|---|---|---|---|
| Female (%) | 19 | (73.1%) | 28 | (87.5%) | 19 | (59.4%) | 66 | (73.3%) |
| Age | 59.7 | (10.7) | 49.5 | (13.4)* | 44.8 | (13.7)** | 50.8 | (14.0) |
| BMI (kg/m²) | 23.4 | (2.2) | 39.3 | (6.4)* | 41.2 | (4.5)** | 35.4 | (9.1) |
| Fasting Blood Glucose (mM) | 5.0 | (0.4) | 4.9 | (0.4) | 5.5 | (0.6)**,^{$} | 5.1 | (0.6) |
| Fasting Plasma Insulin | 6.3 | (2.5) | 12.4 | (4.5)* | 32.2 | (16.1)**,^{$} | 17.7 | (14.9) |
| (mU/mL) | | | | | | | | |
| HOMA-IR | 1.4 | (0.6) | 2.7 | (0.9)* | 8.0 | (4.6)**,^{$} | 4.2 | (4.0) |
| QUICKI | 0.37 | (0.03) | 0.34 | (0.02)* | 0.29 | (0.02)**,^{$} | 0.33 | (0.04) |
| Cardiac frequency | 67.6 | (9.8) | 72.6 | (10.4) n = 30* | 81.5 | (12.1) n = 31**,^{$} | 74.3 | (12.2) n = 87 |
| SAP (mmHg) | 129.6 | (14.4) | 127.3 | (20.6) n = 30 | 134.4 | (14.7) | 130.6 | (17.0) n = 88 |
| DAP (mmHg) | 74.6 | (9.9) | 71.9 | (10.1) n = 30 | 73.3 | (12.1) | 73.3 | (11.0) n = 88 |
| Serum Creatinine (μM) | 65.0 | (13.3) | 64.6 | (11.5) | 68.4 | (14.6) | 66.1 | (13.2) |
| 2 h OGTT Glucose (mM) | NA | | 5.9 | (2.0) n = 15 | 7.4 | (1.7) n = 23^{$} | 6.8 | (2.0) n = 38 |
| 2 h OGTT Insulin (mU/mL) | NA | | 60.0 | (44.4) n = 15 | 160.5 | (119.8) n = 23^{$} | 120.9 | (108.4) n = 38 |
| eGFR (mL/min/1.73 m²) | 97.3 | (19.9) | 97.4 | (18.7) | 102.9 | (23.2) | 99.3 | (20.7) |
| Urine pH | 6.2 | (0.8) | 5.8 | (0.7)* | 5.4 | (5.4)**,^{$} | 5.8 | (0.8) |
| Urine Creatinine (mM) | 8.2 | (8.1) | 12.5 | (5.7)* | 16.3 | (8.8)**,^{$} | 12.6 | (8.2) |
| Glycosuria (μM) | 60.8 | (31.9) | 65.5 | (27.5) | 93.7 | (41.6)**,^{$} | 74.2 | (37.0) |

When we gathered NWIS and OWIS together into the Composite Insulin-Sensitive group (CIS), we still observed no significant differences of SAP, DAP, serum creatinine and eGFR between OWIR and CIS. All other demographic, clinical and biochemical parameters were different between groups (Table 1B).

TABLE 1B
Demographics and clinical characteristics of CIS. Unless otherwise
indicated, data are reported as mean (S.D.). Missing data for
some participants are indicated for each variable. CIS (Composite
Insulin-Sensitive group) is formed by the association of NWIS
(Normal Weight Insulin-Sensitive) and OWIS (Over Weight Insulin-
Sensitive) groups. SAP (Systolic Arterial Pressure); DAP (Diastolic
Arterial Pressure); OGTT (Oral Glucose Tolerance Test); eGFR (estimated
Glomerular Filtration Rate) and BMI (Body Mass Index). Comparison
between groups were performed using t-Student test (p < 0.05
for ^{†} CIS vs OWIR).

| | CIS n = 58 | |
|---|---|---|
| Female (%) | 47 | (81.0%) |
| Age | 54.1 | (13.2) ^{†} |
| BMI (kg/m²) | 32.1 | (9.4) ^{†} |
| Fasting Blood Glucose (mM) | 4.9 | (0.4) ^{†} |
| Fasting Plasma Insulin (mU/mL) | 9.7 | (4.8) ^{†} |
| HOMA-IR | 2.1 | (1.0) ^{†} |
| QUICKI | 0.35 | (0.03) ^{†} |
| Cardiac frequency (bpm) | 70. | (10.3) n = 56 ^{†} |
| SAP (mmHg) | 128.4 | (17.9) n = 56 |
| DAP (mmHg) | 73.2 | (10.4) n = 56 |
| Serum Creatinine (μM) | 64.8 | (12.2) |
| 2 h OGTT Glucose (mM) | 5.9 | (2.0) n = 15 ^{†} |
| 2 h OGTT Insulin (mU/mL) | 60.0 | (44.4) n = 15 ^{†} |
| eGFR (mL/min/1.73 m²) | 93.8 | (12.2) |
| Urine pH | 6.0 | (0.8) ^{†} |
| Urine Creatinine (mM) | 10.6 | (7.1) ^{†} |
| Glycosuria (μM) | 63.4 | (29.4) ^{†} |

uBCAA as Early Biomarker for IR Mass-Screening

Elevated concentrations of fasting bBCAA in insulin-resistant subjects were first described in the late 1960s20. After almost 42 years, elevated bBCAA were identified as early T2D biomarkers in a large longitudinal cohort and confirmed in a second prospective study2. Here, we analysed bBCAA and uBCAA in a non-diabetic prospective cohort. bBCAA and uBCAA were quantified using the standard reference method (LC-MS/MS) and SKC synthetic biochemical network (Table 2A, FIGS. 1B, 1E and 1F).

TABLE 2A
Blood and urine BCAA of participants. Unless otherwise indicated, data are reported
as mean (S.D.). NWIS (Normal-weight insulin-sensitive); OWIS (Overweight insulin-
sensitive); OWIR (overweight insulin-resistant); bBCAA (blood Branched-Chain Amino
acids); uBCAA (urine Branched-Chain Amino acids); SKC (Synthetic Biochemical Network
for BCAA detection). Comparison between groups were performed using t-Student test
(p < 0.05 for *NWIS vs OWIS; **NWIS vs OWIR and ^{$}OWIS vs OWIR).

| | NWIS N = 26 | | OWIS N = 32 | | OWIR N = 32 | | TOTAL N = 90 | |
|---|---|---|---|---|---|---|---|---|
| bBCAA (LC-MS/MS (μM)) | 371.1 | (58.1) | 430.7 | (85.3)* | 521.0 | (94.9)**,^{$} | 445.6 | (101.8) |
| bBCAA (SKC (μM)) | 738.3 | (130.8) | 764.5 | (150.5) | 929.4 | (166.5)**,^{$} | 815.5 | (172.3) |
| uBCAA (LC-MS/MS (μM)) | 51.0 | (24.5) | 96.7 | (35.9)* | 150.5 | (62.7)**,^{$} | 102.6 | (60.0) |
| uBCAA (SKC (μM)) | 52.6 | (20.4) | 85.1 | (31.4)* | 126.0 | (45.8)**,^{$} | 90.3 | (45.5) |

We evaluated the analytical performance of the SKC synthetic biochemical method for uBCAA quantification in comparison with LC-MS/MS (N=90; FIG. 2A). Both methods obtained comparable results with respect to uBCAA concentrations. SKC uBCAA quantification was performed weekly according to patients' recruitment. LC-MS/MS uBCAA method was performed in three independent runs. The Intraclass Correlation Coefficient between both methods is 0.87 (FIG. 2 and FIG. 6).

bBCAA measured by SKC synthetic biochemical network correlated weaker to LC-MS/MS quantification (Intraclass Correlation Coefficient r=0.17, FIG. 6). The bBCAA concentrations measured using the SKC synthetic biochemical network were higher than those measured using LC-MS/MS with a ratio of 1.83. This difference may be explained by the fact that no protein precipitation is performed using SKC method. bBCAA and uBCAA presented a moderate pearson correlation independently of the method used for quantification (r=0.61 and r=0.54 with a bBCAA/uBCAA ratio of 4.34 and 9.03 for LC-MS/MS and SKC, respectively. FIG. 6, Table 2).

In the same line with earlier studies, we show that bBCAA were associated with all insulin resistance indices evaluated in our study (i.e. fasting plasma insulin, HOMA-IR and QUICKI, 2 h insulin following an OGTT; FIG. 6).

Interestingly, uBCAA presented a higher correlation with the same insulin resistance parameters compared to bBCAA (i.e. Pearson correlation coefficient r=0.65 vs r=0.53 for HOMA-IR vs SKC uBCAA or bBCAA, respectively FIG. 6) but this correlation was not homogeneous among groups (Pearson correlation coefficient r=0.68, r=0.48 and r=0.23 for NWIS, OWIS and OWIS, respectively-FIG. 6 and FIGS. 7A-7B). No correlation was not observed between bBCAA and urine creatinine.

Then, we evaluated the possibility of using uBCAA as a diagnostic tool for the detection of insulin-resistant subjects. We used a receiver operating characteristic regression (ROC regression) to compare SKC synthetic biochemical network for uBCAA quantification to HOMA-IR index. uBCAA presented an overall diagnostic accuracy of 88% calculated using the area under curve (FIG. 2B). We evaluated different cut-off points of SKC uBCAA concentration regarding their sensitivity/specificity performances (Table 3). Specificity were from 48.30% to 79.3% and sensitivity from 96.9% to 75.0% for SKC uBCAA cut-offs between 65 μM and 95 μM.

TABLE 2B
Demographics and clinical characteristics of CIS. Unless
otherwise indicated, data are reported as mean (S.D.).
CIS (Composite Insulin-Sensitive group) is formed by the
association of NWIS (Normal Weight Insulin-Sensitive) and
OWIS (Over Weight Insulin-Sensitive) groups. bBCAA (blood Branched-
Chain Amino acids); uBCAA (urine Branched-Chain Amino acids); SKC
(Synthetic Biochemical Network for BCAA detection). Comparison
between groups were performed with Student (p < 0.05 for
^{†} CIS vs OWIR).

| | CIS n = 58 | |
|---|---|---|
| bBCAA (LC-MS/MS - μM) | 404.0 | (79.6) ^{†} |
| bBCAA (SKC - μM) | 752.7 | (141.4) ^{†} |
| uBCAA (LC-MS/MS - μM) | 76.2 | (38.6) ^{†} |
| uBCAA (SKC - μM) | 70.6 | (31.4) ^{†} |

### EXAMPLE 3: INNOVATIVE SIMPLE AND RAPID URINE INSULIN RESISTANCE ASSAY

We adapted an enzymatic test to detect and quantify BCAAs. Briefly, the enzyme L-Leucine Dehydrogenase (LeuDH) produces NADH in the presence of the three BCAA (L-leucine, L-Isoleucine and L-Valine). Then, NADH reduces Thiazolyl Blue Tetrazolium Blue (MTT) via the electron transport mediator 1-Methoxy-5-methylphenazinium methyl sulfate (1M-PMS). The blue intensity of MTT is proportional to BCAA levels present in the samples. This SKC synthetic biochemical network allows uBCAA quantification using existing standard devices in biologic analysis laboratories.

TABLE 3
Diagnostic performances of the SKC synthetic biochemical network method for different
of uBCAA cut-offs. Sensitivity Performances are shown for different thresholds
calculated with HOMA-IR (cut-off > 4.0) as reference diagnostic standard
test. Sensitivity was the proportion of positive index test (defined as uBCAA
concentration above the cut-off) in the insulin resistant population and specificity
the proportion of negative (defined as uBCAA concentration under the cut-off)
in the insulin sensitive population, according to the HOMA-IR index.

| | SKC uBCAA Cut-off | | | | | | |
|---|---|---|---|---|---|---|---|
| | 65 μM | 70 μM | 75 μM | 80 μM | 85 μM | 90 μM | 95 μM |
| Sensitivity | 96.90% | 90.60% | 90.60% | 90.6% | 84.4% | 81.3% | 75.0% |
| Specificity | 48.30% | 55.20% | 60.30% | 69.0% | 70.7% | 74.1% | 79.3% |

Besides, this test can also be adapted to our engineered approach to implement logic gates and build-up programmable synthetic biochemical networks in non-living vesicles for biomarkers detection22. To proof this concept and validate the usability of our approach in a Point of Care setup, we used a homemade microfluidic chip to produce non-living giant unilamelar vesicles (GUVs) containing the SKC synthetic biochemical networks for the detection of either urinary BCAA or glucose (FIG. 4 and FIGS. 8A-8C)).

The rationale for BCAA and Glucose detection in a single test is based on a double opportunity to identify IR and possible diabetes. While high uBCAA would detect IR, high glycosuria could suggest associated diabetes (FIG. 3). High glycosuria should prompt for fasting blood glucose measurement in order to assess the diagnosis of diabetes.

GUVs containing the SCK synthetic biochemical networks for the detection of uBCAA or glucose are then entrapped into macroscopic alginate beads. Each bead contains about 60 000 GUVs and were designed to respond specifically to uBCAA or urine glucose in concentrations above 100 μM. This cut-off was chosen based on the diagnostic performances of IR using SKC synthetic biochemical network for the uBCAA quantification (Table 3). The colored output for uBCAA is given by the reduced MTT (blue color). Glucose detection is performed using the canonical Glucose Oxidase/Peroxidase couple with a violet endpoint from the oxidized form of AmplexRed, resorufin (FIG. 4).

TABLE 4
Normalized excretion of uBCAA to urine creatinine. Data are reported
as mean (S.D.). uBCAA/Creatinine Ratio was calculated dividing
BCAA concentration quantified using SKC method by urine Creatinine
concentration quantified using the Creatinine Assay Kit from Sigma
Aldrich (MAK080), as described in the Methods section.

| uBCAA/Creatinine (μM/mM) | | |
|---|---|---|
| | N = 90 | N = 89^{†} |

| | | | | |
|---|---|---|---|---|
| NWIS | 40.9 | (156.3) | 10.3 | (8.7) |
| OWIS | 8.2 | (4.4) | 8.2 | (4.4) |
| OWIR | 10.3 | (6.6) | 10.3 | (6.6) |
| Total | 18.4 | (84.2) | 9.5 | (6.6) |

^{†}One participant was considered as outlier and was excluded from part of the analysis. The outlier was a participant from NWIS group and presented an urine creatinine concentration of 0.05 mM and an uBCAA concentration of 40.3 μM. Its uBCAA/Creatinine ratio was 806 μM/mM.

Low levels of both biomarkers are expected in healthy condition (FIG. 3). A threshold of 80 μM was determined for our proof of concept and SKC/GUVs respond to BM concentrations above this cut-off (FIG. 4).

GUVs containing the biochemical networks for the detection of uBCAA or glucose are then entrapped into macroscopic alginate beads. Each bead contains about 60 000 GUVs and were designed to respond to a concentration of/about 80 μM. The colored output for uBCAA is given by the reduced MTT (blue color). Glucose detection is performed using the canonical Glucose Oxidase/Peroxidase couple with a violet endpoint from the oxidized form of AmplexRed, resorufin (FIG. 4).

### EXAMPLE 4: DETERMINATION OF SUBSTRATE SPECIFICITY OF THE ENZYME LEUCINE DEHYDROGENASE (LEUDH) FROM B. CEREUS CONTAINING A SUMO-PROTEIN DOMAIN

### Natural Proteinogenic L-Amino Acids as Substrates

The IDIr biochemical network uses the enzyme Leucine Dehydrogenase (LeuDH) for oxidizing the branched-chain amino acids (BCAA) and for producing the colorimetric substrate, blue reduced MTT. The absorbance values are proportional to BCAA levels. Detection and measurement branched-chain amino acids in biological fluids (serum and urine) correlate with insulin-resistance indexes. Thus, the substrate specificity (other L-amino acids than BCAA) of Leucine Dehydrogenase (LeuDH) from B. cereus containing a SUMO-protein domain needs to be verified to avoid erroneous results using IDIr test.

Analysis of the substrate specificity of the Leucine Dehydrogenase co-expressed with SUMO-protein domain was done using 18 natural proteinogenic L-amino acids in the same conditions used for determining the kinetic parameter of LeuDH with BCAA as substrates.

### Objectives

Determine the substrate specificity of Leucine Dehydrogenase co-expressed with SUMO-protein using all the 20 natural proteinogenic amino acids.

### Material

### Reagents and Materials:

Bacillus cereus Leucine Dehydrogenase from Sigma Aldrich (79846-1 mL, lot #BCBN 1697V)
β-Nicotinamide adenine dinucleotide hydrate (NAD+) from Sigma Aldrich (N7004)
Curcubita sp. Ascorbate Oxidase from Sigma Aldrich (A0157-250UN, lot #036M4071V)
1-Methoxy-5-methylphenazinium methyl sulfate from Sigma Aldrich (8640-100 MG, lot #MKBW5046V)
Thiazolyl Blue Tetrazolium Bromide (MTT) from Sigma Aldrich (M2128-500 MG)
L-Leucine from Sigma Aldrich (L8000-25G, lot #BCBQ9986L)
L-Valine from Sigma Aldrich (V0500-25G, lot #SLBK4241V)
L-Isoleucine from Sigma Aldrich (12752-10G, lot #SLBM7711V)
L-Alanine from Sigma Aldrich (05129-25G, lot #XXX)
L-Arginine from Sigma Aldrich (A5006-100G, lot #XXX)
-Aspartate from Sigma Aldrich (A9256-100G, lot #XXX)
L-Glycine from Sigma Aldrich (G7126-100G, lot #XXX)
L-Glutamate from Sigma Aldrich (G1251-100G, lot #XXX)
L-Glutamine from Sigma Aldrich (G3126-100G, lot #XXX)
L-Histidine from Sigma Aldrich (H8000-25G, lot #XXX)
L-Lysine from Sigma Aldrich (62840-25G, lot #XXX)
L-Methionine from Sigma Aldrich (M9625-25G, lot #XXX)
L-Phenylalanine from Sigma Aldrich (78019-25G, lot #XXX)
L-Proline from Sigma Aldrich (81709-25G, lot #XXX)
L-Serine from Sigma Aldrich (S4500-100G, lot #XXX)
L-Threonine from Sigma Aldrich (T8625-25G, lot #XXX)
L-Tryptophan from Sigma Aldrich (T0254-25G, lot #XXX)
L-Tyrosine from Sigma Aldrich (T-3754-50G, lot #XXX)
Trizma© Base from Sigma Aldrich (T1503-1 KG, lot #SLBG2652V)
96 Well Microplate, Solid Bottom, Half Area from Greiner Bio-One (675101, lot #E18013CL)

### Solutions

100 mM Tris-HCl pH7.5 buffer

All solutions were prepared in 100 mM Tris-HCl pH7.5 buffer

### A Solution:

NAD+50 mM
Ascorbate Oxidase 10 U/mL
1M-PMS 0.2 mM

### B Solution

MTT 2 mM
Leucine Dehydrogenase 75 U/mL
L-amino acids
L-Leucine 1 mM
L-Valine 1 mM
L-Isoleucine 1 mM
L-Alanine 1 mM
L-Arginine 1 mM
L-Aspartate 1 mM
L-Glycine 1 mM
L-Glutamate 1 mM
L-Glutamine 1 mM
L-Histidine 1 mM
L-Lysine 1 mM
L-Methionine 1 mM
L-Phenylalanine 1 mM
L-Proline 1 mM
L-Serine 1 mM
L-Threonine 1 mM
L-Tryptophan 1 mM
L-Tyrosine 1 mM

### Methods

8 μL of A Solution were pre-incubated with 53 μL of each L-amino acid separately in a well of the microplate for 5 minutes at 37° C. Then, 4 μL of B Solution were added. The kinetics of the reactions were followed during 30 minutes at 37° C. Absorbances were read at 600 nM every 60 seconds.

Blanks were performed using Tris-HCl 100 mM pH 7.5 buffer containing no L-amino acid during the incubation with A solution. Experiments were performed in duplicates. Results were normalized to the oxidation of L-leucine at 15 minutes.

### Results (See FIG. 10)

Oxidation of L-amino acids was followed by colorimetric reduction of Thyazolyl Blue Tetrazolium Bromide. Absorbance at 600 nm is proportional to the concentration of L-amino acids in solution. Table 5 and FIG. 10 show the measured absorbance for different L-amino acids at 15 minutes of reaction at 37° C.

TABLE 5
Absorbance values for L-amino acids at 15 minutes at 37° C.

| L-amino acid | A 600 nm | ^{a} Normalized activity (%) |
|---|---|---|
| LEU | 0.587 | 100.0 |
| VAL | 0.612 | 104.3 |
| ILE | 0.601 | 102.5 |
| ALA | 0.002 | 0.3 |
| ARG | 0.003 | 0.4 |
| ASP | 0.000 | 0.0 |
| GLY | 0.001 | 0.1 |
| GLU | 0.002 | 0.3 |
| GLN | 0.001 | 0.2 |
| HIS | 0.001 | 0.2 |
| LYS | 0.001 | 0.1 |
| MET | 0.019 | 3.2 |
| PHE | 0.002 | 0.3 |
| PRO | 0.008 | 1.3 |
| SER | 0.001 | 0.2 |
| THR | 0.002 | 0.3 |
| TRP | 0.003 | 0.4 |
| TYR | 0.002 | 0.3 |

^{a}Values were normalized to L-Leucine oxidation at 15 minutes.

### EXAMPLE 5: SKILLCELL (SKC) MICROFLUIDIC METHODS FOR VESICLE PRODUCTION

We developed original approaches for the production of IDIr-containing vesicles. We use microfluidic chips to encapsulate our biochemical networks. Our method allows the optimal encapsulation of IDIr test controlling the content and the composition of SkillCells in a high-throughput manner.

### SKC3.1 Process

This new motif was designed to be more stable regarding the control of the flow of the 3 different inlets (biochemical network, Octanol/LP and external buffer) as well as to allow complete release of octanol excess from vesicles inside the microfluidic chip. Lastly, modifications intended to permit the production of smaller (˜30 μm of diameter) and more stable vesicles. Overview of SKC3.1 design is shown in FIGS. 8A and 8B.

Major Modifications of the Protocol were:
The T-junction was replaced by a flow-focusing to allow better simultaneous control of flows (FIGS. 8A-8B). SKC3.1 uses two consecutive flow focusing regions to produce double-emulsion vesicles. The first flow focusing is spaced 450 μm from the second flow focusing region, which allow complete encapsulation and formation of the first emulsion (w:o) before entering in the second flow focusing region. The simpler geometry of SKC3.1 renders this motif easier to be produced as well as more performant for vesicle production.
DPPC was replaced by DOPC (1,2-dioleoyl-sn-glycero-3-phosphocholine) as phospholipid for the composition of bi-layered membrane. DOPC possesses a lower phase transition temperature (−20° C.) that allows the bi-layered membranes composed of DOPC to be in the liquid phase in temperatures above −20° C. Membranes in liquid phase are more fluid and thus more stable.
All channels were reduced to allow the production of smaller GUV. Smaller vesicles are more stable than bigger ones. SKC3.1 produces vesicles from 15 to 30 μm of diameter;
Vesicle harvest channel was elongated to permit the complete detachment of 2-octanol droplets from vesicles' surface inside the microfluidic chip.
The biochemical network (enzymes and metabolites) was dissolved in Tris-HCl 50 mM pH7.5 without glycerol.
DOPC instead of DPPC was used as phospholipid for the composition and formation of the bilayer membrane of the vesicles at 3.5 mM and was dissolved in 2-octanol.
For vesicles containing BCCA-detection network Cholesterol (CHO) was added at 9:1 (DOPC:CHO) molar ration in Octanol/LP phase.
External phase buffer was 15% (w/v) glycerol and 3.5% (w/v) pluronic F68 in water. Typical flow conditions used with SKC3.1 design are around 65 μL/min for external buffer solution, 2.5 L/min for biochemical network and 0.7 μL/min for oil/lp solutions.

SKC3.1 produces GUV at a frequency of 1 000 hertz. Vesicles produced using SKC3.1 are monodisperse (ranging from 15 μm to 30 μm depending on flow/pressure parameter during the production process).

Vesicles are stable in external phase buffer solution for several days at either room temperature or 4° C. No leakage of encapsulate content was observed over 30 days of storage at RT or 4° C. SKC3.1 was chosen for IDIR-vesicles production. Biochemical networks' composition for glucose or BCAA detection and encapsulate by vesicles are described in table 6.

TABLE 6
Biochemical network composition.

| Test | Reagent | Concentration | |
|---|---|---|---|
| BCAA Detection | LeuDH | 150 | U/mL |
| | Ascorbate Oxidase | 10 | U/mL |
| | NAD⁺ | 50 | mM |
| | 1m-PMS | 0.2 | mM |
| Glucose Detection | Glucose Oxidase | 300 | U/mL |
| | HRP | 300 | U/mL |
| | AmplexRed | 1 | mM |

### CONCLUSION

IR and T2D are known to disturb podocyte and kidney function¹⁶ and urinary amino acid excretion is altered in impaired renal state¹⁷. In this work we analysed only normoglycaemic subjects presenting normal renal function (Stage 1 and 2 of CKD-eGFR>60 mL/min/1.73 m²). The three groups (overweight, insulin-resistant (OWIR); overweight, insulin-sensitive (OWIS); and normal weight, insulin-sensitive (NWIS)) presented similar kidney function despite higher concentrations of uBCAAs in OWIR compared to both insulin sensitive groups (OWIS and NWIS). uBCAA were also higher in OWIS compared to NWIS. uBCAA still associate with IR indices (i.e. fasting plasma insulin, HOMA-IR and QUICKI, 2 h insulin following an OGTT) when considering only Stage 1 of CKD (eGFR≥90 mL/min/1.73 m²; N=56, r=0.69 for SKC uBCAA and HOMA-IR).

A “clogging” model was recently proposed in which different tissues would compensate skeletal muscle dysregulated BCAA disposal40. Following this rational, kidney might compensate raised blood BCAA levels by augmenting the ratio of BCAA clearance. We demonstrated here that bBCAA and uBCAA correlated (r=0.65 for Stage 1 and 2 of CKD (N=90); and r=0.67 for Stage 1 of CKD only (N=56), using LC-MS/MS data for bBCAA and uBCAA quantification) amongst the three groups. However, the ratio bBCAA/uBCAA was almost three times higher in NWIR compared to OWIR in Stage 1 of CKD (10.4; 5.2 and 3.6 for NWIS, OWIS and OWIR, respectively). When Stage 1 and 2 of CKD were analysed, this pattern persisted (9.1; 5.2 and 3.9 for NWIS, OWIS and OWIR, respectively), despite higher bBCAA concentrations in OWIR. Excess circulating bBCAAs appear to be selectively eliminated (Table 4).

Urine specimen has many advantages compared to blood in an IR/CMBCD mass screening context. Self-collecting urine is non-invasive and urinary tests are much less expensive than their blood counterparts. In addition, we show in this work that the association between uBCAA and IR indices (e.g. HOMA-IR and QUICKI) are stronger than those between bBCAA and these same indices. Moreover, our results show that uBCAA is more relevant in diagnosing/predicting IR than bBCAA compared to HOMA-IR index (diagnostic accuracy of 88.8% and 80.3% for uBCAA and bBCAA, respectively). We evaluated different uBCAA cut-offs as predictor tool for IR detection. Good sensitivities (96.9-75.0%) and specificities (48.3-79.3%) were obtained for uBCAAs concentrations between 65 μM-95 μM. In our study, HOMA-IR was used as the reference standard test to settle IR status of subjects, as this index is the most used in clinics.

The identification of uBCAA cut-offs allows the categorisation of IR subjects independently of their BMI and paves the way for and IR assessment based on the quantitative measurement of uBCAA. LC-MS/MS is the most used technology in the quantification of uBCAA. Unfortunately, it is costly and time consuming. In order to be compliant with mass screening constraints and diagnosis excellence, we combined the non-invasive urinary sampling with our method based on synthetic biology principle to design artificial biochemical networks to quantitatively detect uBCAA. Quantification of uBCAA using LC-MS/MS highly correlated with our synthetic biochemical approach and validated our method (Intraclass Correlation Coefficient=0.87, uBCAA LC-MS/MS:uBCAA SKC ratio=1.11). The rapid and simple synthetic biochemical network test we designed can be performed on ubiquitous laboratory equipment like plate readers.

Here, by using a synthetic biochemical network to detect urine BCAA (uBCAA) we show that their concentration are increased in overweight non-diabetic insulin-resistant individuals compared to that of the insulin-sensitive subjects, independently of their body-mass-index (BMI). Individuals with normal kidney function (n=90) recruited at the University Hospital of Montpellier (France) were distributed in three groups according to their BMI and IR states (normal weight, insulin-sensitive (NWIS); overweight insulin-sensitive (OWIS); and overweight insulin-resistant (OWIR). We found that uBCAA levels were significantly higher in OWIR group compared to OWIS and NWIS (148.2 μM; 100.5 μM; and 51.0 μM, respectively). uBCCA levels were still significantly higher in OWIR when compared to the gathered composite insulin-sensitive individuals (CIS-78.7 μM). We present here a proof of concept of a fast and reliable test for the screening of IR based on the detection of uBCAA. Our results demonstrate that uBCAA can identify insulin-resistant status among overweight persons. Their simplified quantification using our lab-free approach and appropriate thresholds could allow action for an effective reduction of risk for T2D and cardiovascular disease.

In addition, our artificial biochemical networks can also be adapted to a lab-free technology. The synthetic biochemical construction can be encapsulated into artificial bilayer membranes and can be set to return visible outputs in few minutes following the detection of the chosen biomarkers in concentrations above the predefined threshold (a so-called SkillCell® biomachine). Confining enzymatic reactions into spatially defined volumes is advantageous. The reactions are usually more timely and efficient. Moreover, bilayer membranes isolate and protect enzymes from inhibitors and disturbers present in complex biological matrices (e.g. blood, urine and saliva). Skillcells® can in turn be entrapped in a controlled fashion into alginate bead structures. At this point, each alginate bead works independently as an uBCAA assay and can be used directly into raw urine samples. This method exempts the need of pre-treatment of urines for uBCAA detection. In few minutes, a visual output appears if the uBCAA threshold is exceeded. The test can performed during the clinical consultation by a trained staff. In few minutes, a visual output appears if the uBCAA threshold is exceeded. This is the first proof of concept of a non-invasive rapid and simple IR assay compliant with clinical constrains of mass population screening campaigns at a reversible stage of T2D onset.

In our clinical study, blood and urine samples from normoglycaemic, normal weight/overweight and insulin sensitive/resistant subjects were evaluated. Compared to HOMA-IR index, the detection of uBCAA using our approach presented an accuracy of 88% for the diagnosis of IR. The easiness of using urine samples for the detection of high levels of BCAA in large screening campaigns for T2D would be beneficial for patients at risk of T2D. We also present here a proof of concept of an engineered simplified rapid IR test using uBCAA using DNA free synthetic biology principles which is able to provide a colorimetric output in response to an uBCAA threshold.

In conclusion, we showed here that uBCAA are augmented in IR and that they can be used as biomarkers for IR and CMBCD despite the BMI of the patients. We also proposed a reliable method for uBCAA quantification and a threshold capable to differentiate IR from IS patients. Lastly, we presented the first proof of concept of a non-invasive rapid and simple IR assay compliant with clinical constrains of mass population screening campaigns for identification of increased risk of cardiometabolic disorders, and focused lifestyle interventions to reduce their emergence.

### BIBLIOGRAPHY

1 Wang, T. J. et al. Metabolite profiles and the risk of developing diabetes. Nat Med 17, 448-453, doi: 10.1038/nm. 2307 (2011).
2 Jang, C. et al. A branched-chain amino acid metabolite drives vascular fatty acid transport and causes insulin resistance. Nat Med 22, 421-426, doi: 10.1038/nm. 4057 (2016).
3 Newgard, C. B. Interplay between lipids and branched-chain amino acids in development of insulin resistance. Cell Metab 15, 606-614, doi: 10.1016/j.cmet.2012.01.024 (2012).
4 Newgard, C. B. et al. A branched-chain amino acid-related metabolic signature that differentiates obese and lean humans and contributes to insulin resistance. Cell Metab 9, 311-326, doi: 10.1016/j.cmet.2009.02.002 (2009).
5 Haufe, S. et al. Branched-chain and aromatic amino acids, insulin resistance and liver specific ectopic fat storage in overweight to obese subjects. Nutr Metab Cardiovasc Dis 26, 637-642, doi: 10.1016/j.numecd.2016.03.013 (2016).
6 Lee, C. C. et al. Branched-Chain Amino Acids and Insulin Metabolism: The Insulin Resistance Atherosclerosis Study (IRAS). Diabetes Care 39, 582-588, doi: 10.2337/dc15-2284 (2016).
7 Wiklund, P. et al. Insulin resistance is associated with altered amino acid metabolism and adipose tissue dysfunction in normoglycemic women. Sci Rep 6, 24540, doi: 10.1038/srep24540 (2016).
8 Wang, Q., Holmes, M. V., Davey Smith, G. & Ala-Korpela, M. Genetic Support for a Causal Role of Insulin Resistance on Circulating Branched-Chain Amino Acids and Inflammation. Diabetes Care 40, 1779-1786, doi: 10.2337/dc17-1642 (2017).
9 Palmer, N. D. et al. Metabolomic profile associated with insulin resistance and conversion to diabetes in the Insulin Resistance Atherosclerosis Study. J Clin Endocrinol Metab 100, E463-468, doi: 10.1210/jc.2014-2357 (2015).
10 Crossland, H. et al. Exploring mechanistic links between extracellular branched-chain amino acids and muscle insulin resistance: an in vitro approach. American journal of physiology. Cell physiology 319, C1151-c1157, doi: 10.1152/ajpcell.00377.2020 (2020).
11 White, P. J. & Newgard, C. B. Branched-chain amino acids in disease. Science 363, 582-583, doi: 10.1126/science.aav0558 (2019).
12 Courbet, A., Amar, P., Fages, F., Renard, E. & Molina, F. Computer-aided biochemical programming of synthetic microreactors as diagnostic devices. Mol Syst Biol 14, e7845, doi: 10.15252/msb.20177845 (2018).
13 Courbet, A., Molina, F. & Amar, P. Computing with synthetic protocells. Acta Biotheor 63, 309-323, doi: 10.1007/s10441-015-9258-8 (2015).
14 Courbet, A., Renard, E. & Molina, F. Bringing next-generation diagnostics to the clinic through synthetic biology. EMBO Mol Med 8, 987-991, doi: 10.15252/emmm.201606541 (2016).
15 Santos Schneider, F. et al. Biomachines for Medical Diagnosis. Advanced Materials Letters 11, 1-5, doi: 10.5185/amlett.2020.041499 (2020).
16 Welsh, G. I. et al. Insulin signaling to the glomerular podocyte is critical for normal kidney function. Cell Metab 12, 329-340, doi: 10.1016/j.cmet.2010.08.015 (2010).
17 Masania, J. et al. Urinary Metabolomic Markers of Protein Glycation, Oxidation, and Nitration in Early-Stage Decline in Metabolic, Vascular, and Renal Health. Oxidative medicine and cellular longevity 2019, 4851323, doi: 10.1155/2019/4851323 (2019).
18 Zhou, M. et al. Targeting BCAA Catabolismo Treat Obesity-Associated Insulin Resistance. Diabetes 68, 1730-1746, doi: 10.2337/db18-0927 (2019).
19 Akbarzadeh A, Rezaei-Sadabady R, Davaran S, et al. Liposome: classification, preparation, and applications. Nanoscale Res Lett. 2013; 8 (1): 102. Published 2013 Feb. 22. doi: 10.1186/1556-276X-8-102.

1. An in vitro method of determining whether a subject is at risk of being developing or to develop insulin resistance/future Type 2 diabetes (T2D) and/or cardiovascular diseases CVD/for advance alert of T2D and/or CVD onset/for the detection of insulin-resistant subjects at risk of early T2D and/or CVD onset/for the diagnostic and monitoring of insulin-resistance individual from an insulin-sensitive one, the method comprising:
a) from an urine sample obtained from the subject, measuring/determining the concentration of branched-chain amino acids (BCAAs) present in said urine sample (uBCCAs) by an enzymatic reaction network by gathering the urine sample with a solution containing at least Leucine dehydrogenase (LeuDH) enzyme;
and
determining whether the subject is at said risk, wherein a uBCCAs concentration superior or egal to a cut-off (threshold)), is indicative that the subject is at risk of developing or develop insulin resistance/future T2D and/or CVD/can distinguish insulin-resistant individuals from insulin-sensitive ones/for advance alert of T2D and/or CVD onset/for the detection of insulin-resistant subjects at risk of early T2D and/or CVD onset.

2. A method or a process of determining the need or deficiency of a dietary/nutritional supplement for a subject or to analyze the nutritional needs or deficiency of a subject by considering a combination of various health and performance factors (health profile);
comprising:
a) from an urine sample obtained from the subject, measuring/determining at least the concentration of branched-chain amino acids (BCAAs) present in said urine sample (uBCCAs) by an enzymatic reaction network by gathering the urine sample with a solution containing at least Leucine dehydrogenase (LeuDH) enzyme;
b) comparing the result obtained in step a) to the concentration of uBCAAs present in a subject exhibiting a normal health profile and/or control health profile(s) known to require a personalized regimen;
c) determining whether the subject is in need or deficiency of nutritional/dietary supplement(s) and/or requires a personalized regimen or therapy wherein the presence of a uBCCAs concentration superior or egal to a cut-off (threshold)), is indicative that the subject is in need or deficiency of dietary/nutritional supplement(s) and/or personalized regimen or therapy;
d) optionally, preparing a personalized regimen for the subject, the regimen including a customized nutritional formula and/or synergistic physical program and/or personalized therapy; and
administering the regimen to the subject and/or performing the synergistic physical program and/or the personalized therapy over a period of time;
e) optionally, further comprising re-analyzing the subject's needs or deficiencies and, if necessary, adjusting the regimen or the therapy.

3. The method according to claim 1, wherein the uBCAAs cut-off (threshold) is between 65 pM and 95 pM, preferably is between 7 OpM and 90 pM, between 75 pM and 85 pM, more preferably 80 pM.

4. The method according to claim 1, wherein the uBCAAs cut-off used to determine the risk for the subject is the same for a subject obese or not.

5. A method according to claim 1, wherein in step a), the measure/determination of the concentration of uBCAAs is carried out by a method comprising the steps of:
a1) bringing into contact said urine sample with a solution containing Leucine dehydrogenase (LeuDH), P-Nicotinamide adenine dinucleotide hydrate (NAD+) and Thiazolyl Blue Tetrazolium Bromide (MTT);
a2) incubating the composition obtained in step a1);
a3) measuring the output signal generated at step a2); and
a4) determining from said output signal the concentration of uBCCAs.

6. A method according to claim 1, wherein in step a), the measure/determination of the concentration of uBCAAs is carried out by a method comprising in step a), a preliminary step wherein the urine sample of the subject is pre-incubated with ascorbate oxidase in order to eliminate the ascorbic acid, preferably at 37°, preferably in presence of 1-Methoxy-5-methylphenazinium methyl sulfate (1M-PMS), more preferably at about 0.04 mM 1M-PMS.

7. A method according to claim 6, wherein in step a), the LeuDH and ascorbate oxidase enzyme are in solution in 3-(N-morpholino) propanesulfonic acid (MOPS) buffer, preferably in 200 mM MOPS buffer at pH 8.0.

8. The method according to claim 5, wherein in step a1), the LeuDH enzyme is selecting from the group consisting of Bacillus cereus LeuDH, Bacillus stearothermophillus LeuDH, Bacillus cereus LeuDH linked to a SUMO protein group and Bacillus stearothermophillus LeuDH linked to a SUMO protein domain, Bacillus stearothermophillus optionally linked to a SUMO domain being preferred.

9. The method according to claim 1, wherein the method comprises a step b) of determining the concentration of glucose present in said sample, said glucose determination being preferably carried out by an enzyme reaction in presence of glucose oxidase (GO), preferably GO and horse radish peroxidase (HRP) enzyme and Amplex red, more preferably in MOPS buffer, preferably in order to control the fasting of the subject.

10. The method according to claim 9, wherein in step a), the measure/determination of the concentration of uBCAAs) and in step b) the measure of concentration of glucose are carried out on a sample from a fasted subject.

11. The method according to claim 9, wherein:
in step a) and if performed in step b), the two samples are urine sample from the subject; or
in step a) the sample is urine sample and in step b) a blood sample from the subject.

12. The method according to claim 9, wherein:
in step a) and, in step b), the samples are urine samples and the measure/determination of the concentration of uBCAAs and the glucose are carried out on two distinct samples from the subject.

13. The method according to claim 1, wherein:
in step a) the solution containing at least LeuDH enzyme (uBCAAs biochemical network) is encapsulated in a vesicle system, preferably encapsulated within a liposome, a droplet, a polymeric support with selective permeability, more preferably within bilipidic membrane vesicles or unilamellar membrane vesicles, more preferably within giant unilamellar vesicles (GUV), within small unilamellar vesicles or sonicated unilamellar vesicles” (SUV) or within large unilamellar vesicles (LUV).

14. The method according to claim 1, wherein:
in step a) the solution containing at least LeuDH enzyme (uBCAAs biochemical network) is encapsulated in a vesicle system, preferably encapsulated within a liposome, a droplet, a polymeric support with selective permeability, more preferably within bilipidic membrane vesicles or unilamellar membrane vesicles, more preferably within giant unilamellar vesicles (GUV), within small unilamellar vesicles or sonicated unilamellar vesicles” (SUV) or within large unilamellar vesicles (LUV); and
in step b), the solution containing at least the glucose oxidase enzyme (glucose biochemical network) is encapsulated in a vesicle system, preferably encapsulated within a liposome, a droplet, a polymeric support with selective permeability, more preferably within a bilipidic membrane or within an unilamellar membrane, preferably within GUV, within SUV or within LUV, preferably within the same vesicle system as for LeuDH enzyme biochemical network.

15. The method according to claim 13, wherein, the vesicle system is produced by microfluidic process.

16. The method according to claim 13, wherein, the vesicles are giant unilamellar vesicle (GUV) produced by microfluidic process, preferably by using the process comprising the steps of:
a) After etching, silicon wafers are coated with a photoresistant layer (50 pm) and baked. Photolithography performed at 375 nm removes the unexposed resist to reveal microstructures: b) Soft lithography of microfluidic chips was performed using polydimethylsiloxane (PDMS) to produce to microfluidic chip. PDMS microfluidic devices were treated with PVA and the GUV production was based on octanol-assisted liposome assembly (OLA);
c) The formation of vesicles on-chip was controlled via a pressure-driven pump by which flow rates of all three phases (inner aqueous (IA), intermediary lipid-octanol oil (LO) and outer aqueous (OA)) were tuned in real-time,
The first emulsion (water-in-oil; W/O) was generated in the first flow-focusing motif where LO wets the hydrophobic PDMS walls and surrounds IA phase, forming spontaneously an internal IA stream and a thin layer of LO phase between PDMS and IA:
In the second flow-focusing region, the OA phase is pumped in a high pressure provoking a shear stress and the pinch-off of the first emulsion W/O,
these steps resulting in a double emulsion (water-in-oil-in-water; W/O/W), wherein:
phospholipids present in the LO phase spontaneously assemble along both water interfaces while the octanol-1 pockets are extracted to form GUVs, and
flows were in the range of 0.5-3 pL/min for IA, 0.2-2 pL/min for LO and 10-120 pL/min for OA. OA to IA flow ratio allows to set the size of the produced GUV (15-75 pm diameter) and the frequency of the production (3 000-10 000 Hz).

17. The method according to claim 13, wherein:
in step a) and b) the vesicle containing the LeuDH biochemical network and the vesicle containing the glucose oxidase biochemical network are entrapped into polymeric matrices “like” hydrated alginate gels, and solid or semi-solid matrices.

18. A kit comprising:
Ascorbate oxidase and LeuDH, preferably in solution in MOPS; and optionally
Glucose oxidase and HRP, preferably in solution in MOPS.

19. A kit comprising:
Ascorbate oxidase and LeuDH in solution in MOPS; and
Glucose oxidase and HRP in solution in MOPS.

20. A kit comprising:
Ascorbate oxidase and LeuDH, preferably in solution in MOPS, encapsulated in a vesicle system, preferably in GUV, SUV or LUV vesicles, preferably GUV obtained by microfluidic process, more preferably by the process of claim 16; and optionally
Glucose oxidase and HRP, preferably in solution in MOPS, encapsulated in a vesicle system, preferably in GUV, SUV or LUV vesicles, preferably GUV obtained by microfluidic process, more preferably by the process of claim 16.

21. A kit comprising:
Ascorbate oxidase and LeuDH, preferably in solution in MOPS encapsulated in a vesicle system, preferably in GUV, SUV or SUV vesicles, preferably GUV obtained by microfluidic process, more preferably by the process of claim 16; and
Glucose oxidase and HRP in solution in MOPS encapsulated in a vesicle system, preferably in GUV, SUV or LUV vesicles, preferably GUV obtained by microfluidic process, more preferably by the process of claim 16.

22. Kit A kit comprising beads, preferably alginate beads, wherein the beads contain:
Ascorbate oxidase and LeuDH, preferably in solution in MOPS encapsulated in a vesicle system, preferably in GUV, SUV or LUV vesicles, preferably GUV obtained by microfluidic process, more preferably by the process of claim 16; and optionally
Glucose oxidase and HRP in solution in MOPS encapsulated in a vesicle system, preferably in GUV, SUV or LUV vesicles, preferably GUV obtained by microfluidic process, more preferably by the process of claim 16.

23. Kit comprising beads, preferably alginate beads, wherein the beads contain: —Ascorbate oxidase and LeuDH in solution in MOPS encapsulated in a vesicle system, preferably in GUV, SUV or LUV vesicles, preferably GUV obtained by microfluidic process, more preferably by the process of claim 16; and
Glucose oxidase and HRP in solution in MOPS encapsulated in a vesicle system, preferably in GUV, SUV or LUV vesicles, preferably GUV obtained by microfluidic process, more preferably by the process of claim 16.

24. A method for the production of GUV vesicles comprising the steps a, b) and c) as defined in claim 16.
